(19) United States
(12) Patent Application Publication
(10) Pub. No.: US 2025/0186048 A1
(43) Pub. Date: Jun. 12, 2025

(54) Better Sealing for Implantable Devices

(71) Applicant: Motaz Shaher, Fort Wayne, IN (US)

(72) Inventor: Motaz Shaher, Fort Wayne, IN (US)

(21) Appl. No.: 18/977,568

(22) Filed: Dec. 11, 2024

Related U.S. Application Data
(60) Provisional application No. 63/608,540, filed on Dec. 11, 2023.

(51) Int. Cl. A61B 17/12; A61B 17/00; A61F 2/24

(52) U.S. Cl. CPC A61B 17/12031; A61B 17/12122; A61B 17/12131; A61F 2/2409; A61B 2017/00867; A61B 2560/04; A61F 2210/0014

(57) ABSTRACT

The present invention relates to an implantable device comprising a skeleton, and an attached covering material to enable better sealing and/or anchoring to address the issue of mismatch between the shape of the implantable device when fully extended and an opening of any kind it is made to occlude. Alternatively, the implantable device as disclosed can be used to generate a prosthetic valve. The present invention utilizes one or more protrusions/extensions with or without additional attached or linked covering material, and the protrusions and covering material when combined with an attachment ring can form a unit called a Sealing Halo unit. Any combination of protrusions and the covering material can extend into any gap on the outside of the periphery of the implantable device. The implantable device can generate various types of implantable medical devices including Left Atrial Appendage (LAA) occluders, Transcatheter Aortic Valve Replacement (TAVR), Transcutaneous Mitral Valve Replacement, Atrial Septal Occluders, Patent Foramen Ovale occluders, Vascular Plugs, etc. In addition, the disclosed implantable device can also generate non-medical devices to occlude any kind of non-medical openings.

### PRIORITY

This application claims priority for the U.S. provisional application Ser. No. 63/608,540 filed on Dec. 11, 2023.

### FIELD OF THE INVENTION

The present invention generally relates to apparatus, systems, devices, and methods for producing and implanting implantable devices. More specifically, it relates to apparatus, systems, devices, and methods for producing and implanting implantable devices with improved and better sealing.

### INCORPORATION BY REFERENCE

All publications, patents, and patent applications mentioned in this specification are herein incorporated by reference in their entirety to the same extent as if each publication or patent application was specifically and individually indicated to be incorporated by reference.

### BACKGROUND OF THE INVENTION

Atrial fibrillation is a condition in which the normal beating of the left atrium (LA) is chaotic and ineffective. The left atrial appendage (LAA) is a blind pouch of the LA. In patients with Atrial fibrillation blood stagnates in the LAA facilitating clot formation. These clots (or clot fragments) tend to embolize or leave the LAA and enter the systemic circulation. A stroke occurs when a clot/clot fragment embolizes and occludes one of the arteries perfusing the brain. Anticoagulants, e.g., Coumadin, have been shown to significantly reduce the stroke risk in Atrial fibrillation patients. These drugs reduce clot formation but also increase bleeding complications including hemorrhagic strokes, subdural hematoma, and bleeding in the gastrointestinal tract.

There are about eight million people in the USA and EU with Atrial fibrillation. About 4.6 million of these patients are at high risk for stroke and would benefit from anticoagulation. However, a large portion of these patients cannot take anticoagulants due to an increased bleeding risk, leaving their stroke risk unaddressed. The prevalence of Atrial fibrillation increases with age.

LAA occluder is one example of an implantable medical device in use for Atrial fibrillation patients. It is implanted in the heart in order to prevent stroke in patients with Atrial fibrillation. The device works by occluding the opening of the LAA, which is the part of the heart where most of the blood clots are formed.

There are several LAA occluder devices in use, but the basic design is the same, and it consists of a self-expanding metallic skeleton made from Nitinol wires covered with a biocompatible mesh. The device is inserted into the body in a compressed form inside a catheter, then once it is released, it unfolds (self-expands) into a circular or cylindrical occluder to plug the LAA and seal its opening, thus preventing blood clots from moving from the LAA into the bloodstream. There are two goals with the use of the LAA occluder: sealing the LAA opening; and anchoring it in place so that it does not embolize or move.

However, the existing LAA occluding devices have drawbacks. For instance, although, the existing devices are offered in many sizes, they must be closely matched to the highly variable LAA anatomy of the specific person. This is difficult to do using fluoroscopy and often requires adjunctive imaging in the form of transesophageal echocardiography (TEE), cardiac computed tomography (CT), and magnetic resonance imaging (MRI), all with three-dimensional reconstructions. If the device is significantly oversized, the LAA ostium may become overstretched which leads to tearing, resulting in bleeding into the pericardial space. If the device is too small, it will not adequately seal the LAA ostium and may be prone to embolization. Even if sized correctly, the device forces the oval LAA ostium to take the round shape of the device, often resulting in residual leakage at the edges due to poor sealing.

Furthermore, the existing devices require sufficient spring force or stiffness to seal and anchor to surrounding tissue. If too stiff, these devices may lead to leaking of blood through the tissue into the pericardial space which may lead to cardiac tamponade. Also, the geometry of these devices limits repositioning once the implant is fully expanded. Existing devices also complicate delivery by requiring positioning in the LAA coaxial to the axis of the LAA. Hence, as captured above, there is a problem with the existing devices.

Most implantable medical devices have metallic skeletons that typically consist of a wired frame, which is made using memory metal such as Nitinol, etc. The wires are connected to each other, so they work mostly as a single unit when the device unfolds. This skeleton is typically covered with a biocompatible semi-porous membrane. The implantable device is usually inserted into the body in a compressed form inside a catheter, then the device is slowly released from the catheter into the target location (cavity, body aperture, organ, vessel, etc.) under X-ray guidance. The body of the implantable device, the entire or part of the implantable device, and/or its underlying skeleton then expand/unfold and assume a predetermined shape, given the memory properties of the device's skeleton.

These implantable devices usually assume a cylindrical shape when they are released, unless, there is an external structure that impedes their complete unfolding. While this design is highly efficient, it has limited adaptability to the surrounding tissue (due to the lack of independent motion of the underlying skeleton). If the device is undersized with respect to the target aperture, cavity, or vessel, that needs to be occluded, then the device will be loose, it will not be able to stay in place, and thus, it will increase the risk of embolization. On the other hand, if the implantable device is significantly oversized, to completely fill the aperture, cavity, or vessel, then, the device can cause perforation or compression of adjacent organs. Hence, there is a need for better-suited devices to overcome the discussed problems.

In addition to the above, typically, these implantable devices are not flexible enough to fully occlude odd-shaped spaces or fill small crevices because of their pre-determined shape. Based on the existing designs, complete sealing is typically achieved by forcing the target vessel, aperture, or cavity to assume the shape of a fully deployed implantable device, which typically is circular, just like the implantable device itself, rather than having the implantable device adapt to the shape of the target organ. Such a design therefore creates further issues.

Relatedly, one of the main issues with the LAA occluders is that most of the time, the LAA opening is oval or irregular in shape, while a fully expanded LAA occluder is typically circular in shape, thus gaps can form between the LAA occluder and the inner wall of the LAA (FIGS. 1, and 2). These gaps can let blood clots escape from the distal part of the LAA, then around the LAA occluder through one of these gaps, then into the left atrium which can cause a stroke. FIG. 1 highlights the problem by illustrating a conventionally available left atrial appendage (LAA) Occluder from a side view including an LAA Occluder (2) and the skeleton of the LAA Occluder implanted inside the LAA (1) occluding its opening. FIG. 2 further underscores the aforementioned problem by illustrating a schematic of a conventionally available left atrial appendage (LAA) Occluder from the top view of the LAA Occluder inside the LAA, showing the wall of the LAA (1), the LAA Occluder (2), showing the problem of a gap (3) formed between the LAA Occluder and the inner wall of the LAA and the width of the gap (4), which can be variable. The illustration also shows that one or more gaps can form between the LAA Occluder and the LAA itself. This illustrates the problem where these gaps prevent the LAA Occluder/implantable device from completely sealing the LAA, which can result in clots escaping from the LAA into the systemic circulation, causing a possible stroke.

The above-discussed issue of the mismatch between the shape of a fully-expanded LAA occluder, and the LAA cavity that needs to be sealed by it, represents a major problem that is also seen with other implantable medical devices such as the transcatheter aortic valve replacement (TAVR) (FIG. 3), transcatheter mitral valve replacement, transcatheter tricuspid valve replacement, vascular plugs, atrial septal occluders, etc. For example, gaps can form between the TAVR and the aorta where TAVR is implanted. These gaps can cause leakage of blood around the TAVR which can cause heart failure, among other complications. FIG. 3 illustrates the problem in the art by illustrating a side view of a conventionally available Transcatheter Aortic Valve Replacement (TAVR) (2) with the skeleton of the TAVR (3) implanted inside an aorta (1), and further shows the gap between the body of the TAVR and the aorta (4), from where blood and blood clots can leak.

Therefore, there is a need for an improved LAA occlusion device, especially one that would provide improved and better sealing addressing the above-discussed problems with the existing implantable devices. The present invention addresses this need and problem by providing an improved LAA occlusion device that provides proper sealing referred to as a Halo seal.

### SUMMARY OF THE INVENTION

The following listing of embodiments is a nonlimiting statement of various aspects of the invention. Other aspects and variations will be evident in light of the entire disclosure.

The present invention provides better implantable devices, including implantable medical devices, and non-medical devices for use in certain functions such as occluding an aperture or cavity in the body or functioning as a prosthetic valve, etc.

An aspect of the present invention is to provide an implantable device for occluding an aperture or cavity in the body of an animal, wherein the device is a medical device, wherein the animal is a mammal, and wherein the mammal is human.

An aspect of the present invention provides an implantable device as a replacement for a native valve in the body of an animal, wherein the device is a medical device, wherein the medical device is a prosthetic valve, wherein the animal is a mammal, and wherein the mammal is human.

An aspect of the present invention provides an implantable device for occluding an aperture or cavity in an apparatus, wherein the apparatus comprises a leaking oil pipe or water pipe.

Other objects, features, and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The accompanying drawings are included to provide a further understanding of the invention, and are incorporated in and constitute a part of the present invention and, together with the description, serve to explain the principle of the invention.

In the drawings,

FIG. 1 illustrates a schematic of a conventionally available left atrial appendage (LAA) Occluder from a side view including an LAA Occluder (2) and the skeleton of the LAA Occluder implanted inside the LAA (1) occluding its opening.

FIG. 2 illustrates a schematic of a conventionally available left atrial appendage (LAA) Occluder from the top view of the LAA Occluder inside the LAA, showing the wall of the LAA (1), the LAA Occluder (2), showing the problem of a gap (3) formed between the LAA Occluder and the inner wall of the LAA and the width of the gap (4), which can be variable. The illustration also shows that one or more gaps can form between the LAA Occluder and the LAA itself.

FIG. 3 illustrates a schematic of a side view of a conventionally available Transcatheter Aortic Valve Replacement (TAVR) (2) with the skeleton of the TAVR (3) implanted inside an aorta (1). It also shows the gap between the body of the TAVR and the aorta (4), from where blood and blood clots can leak.

FIG. 4 illustrates an embodiment of the present invention and exemplifies an implantable medical device (1) that is deployed (i.e., unfolds when deployed) inside a target vessel, aperture, or cavity. As shown in the illustration, gaps (3) and (4) can form between the deployed implantable device (1) and the wall of the target organ (2) and (5). Protrusions (shown as parallel lines when unfolded and lumps/flanges when compressed) are attached to the body of the implantable medical device or its skeleton. Protrusions can have independent motion to unfold into the gaps (3) and (4) between the implantable device and the target organ wall. Protrusions do not unfold (as seen with unfolded lumps/flanges (6)) if there is no gap adjacent to them, so they stay compressed (folded) adjacent to the body of the implantable medical device. Because these protrusions are flexible or semi-flexible and possess an independent motion, they can either unfold completely (as seen in the gaps (3) and (4) represented by long parallel lines) or partially (as seen in the gap (4) represented by a shorter parallel line (7) at the end of the said gap (4)), depending on how much gap is present between the implantable device (1) and the target organ wall (2) and (5). Covering material (8) is attached to or supported by the protrusions. Covering materials are attached to or supported by the protrusions and unfold partially or completely based on the unfolding of the protrusions.

FIG. 5 illustrates an embodiment of the present invention and exemplifies an implantable medical device body (1) supported by a skeleton (2). Protrusions (3) are attached to the body or the skeleton of the medical device. Covering material (4) is attached to or supported by the protrusions, and the covering material can move with the protrusions. Here an example of the functional part of the implantable medical device (1) embodiment of the present invention is shown for a valve leaflet (5).

FIG. 6 illustrates an embodiment of the present invention and exemplifies in panel (A) that the mechanism by which the implantable device (2) of the present invention is delivered is as compressed inside a delivery catheter (1). The protrusions (3) and associated covering material (4) are folded or compressed alongside the compressed medical device (2). In panel (B), the same implantable device (2) is shown after being released from the delivery catheter. The protrusions (3) will unfold if there is a sufficient gap(s) around the implantable device. Covering material (4) that is attached to the protrusions will also unfold whenever the protrusions that they are attached to move too.

FIG. 7 illustrates an embodiment of the present invention and exemplifies an implantable device showing the implantable device's skeleton (1) along with the body (2) of the implantable device with protrusions (3) attached to specific locations on the skeleton (1) of the implantable device (1), and other protrusions (4) can also be attached to the body (2) of implantable devices.

FIG. 8 illustrates an embodiment of the present invention and exemplifies an implantable device showing that the protrusions (3) can be an extension of a wire (2) (or other components) of the skeleton (1) of the implantable device.

FIG. 9 illustrates an embodiment of the present invention and exemplifies an implantable device showing various forms and styles of protrusions (2), (3), (4), and (5) over the skeleton (1) of the implantable device, including thin protrusions (3), thicker protrusions (2), thicker and longer protrusions (4), and longer protrusions with variable thickness (5).

FIG. 10 illustrates an embodiment of the present invention and exemplifies a single protrusion magnified on an implantable device showing a specific example of a protrusion (1) with variable thickness (5) and width (2), variable material composition (3), and combination of variable material composition and other physical properties such as width (4).

FIG. 11 illustrates an embodiment of the present invention and exemplifies an implantable device showing in panel (A) protrusions (2) and (3) attached to the skeleton (1) of the implantable device. The protrusions (2) have a similar orientation and their orientation alternates in pattern with another group of protrusions (3). The protrusions (2) have similar width and length, but they alternate in a pattern and orientation with the other group of protrusions (3). In panel (B), another embodiment of the implantable device of the present invention is shown with the skeleton (1) of the implantable device, where protrusions (2) and (3) are attached to the implantable device with different lengths and widths in a pattern, for instance, a wide and short protrusion (3) followed by a narrow and longer protrusion (2) in a pattern in the same orientation.

FIG. 12 illustrates an embodiment of the present invention and exemplifies an implantable device showing protrusions (2), (3), and (4) and the skeleton (1) of the implantable device. Various shapes or lengths of protrusions are attached to the skeleton (1) of the implantable device in various patterns including long, medium, and short lengths (2). This pattern can repeat itself one or more times (3). The protrusions can be attached in different patterns to the skeleton of the implantable device orientation and alternate in a pattern with other protrusion groups (3) and (4).

FIG. 13 illustrates an embodiment of the present invention and exemplifies an implantable device showing varying lengths of distance (1) to (6) between protrusion attachment sites on the skeleton of the implantable device. Exemplified distance between two protrusion attachment sites (1) can be the same as the distance between a separate set of two protrusions (4), or it can be different such as for different distances (2) and (3) between two other protrusion sets; or distance between the body of the protrusions (5) or various parts of the protrusions (6). Variations in distance between the attachment sites and/or the various parts of the unfolded or folded segments of the protrusions such as (1), (2) (3), (4), (5), and (6) can have a distinct pattern.

FIG. 14 illustrates an embodiment of the present invention and exemplifies an implantable device showing a magnified illustration of an attachment site of a protrusion (3) attached to a part of the skeleton (1) of the implantable device of the present invention. The protrusion (3) can assume different shapes when folded or during unfolding. For example, in this illustration, it is curved, and it may have a hook (4) or other variations to enable the attachment of the covering material or to confer additional functions. The width (2) of the attachment site can be variable or patterned along with the angle (5) between the skeleton (1) of the implantable device and the protrusion (3).

FIG. 15 illustrates an embodiment of the present invention and exemplifies an implantable device showing: in the top panel a magnified illustration of a protrusion (2) attached to a part of the skeleton (1) of the implantable device. One or multiple flexible segments (3) and (4) within the protrusion (2) can be made from the same material as the rest of the protrusion (2), but they can be made more flexible because they have different physical properties such as being thinner, or narrower, etc., so they can bend more, or flex more, etc. These segments (3) and (4) can alternatively be made from other materials that are different from the rest of the protrusion (2), wherein these different materials give additional flexibility. In the bottom panel, another embodiment of the implantable device of the present invention is illustrated showing a protrusion (2) attached to a part of the skeleton (1) of the implantable device, the protrusion (2) comprising a mechanical joint (5) which allows additional flexibility and bending.

FIG. 16 illustrates an embodiment of the present invention and exemplifies an implantable device showing a magnified illustration of a protrusion (2) and a part of the skeleton (1) of the implantable device, where the protrusion has one or more hooks or other forms of mechanical attachment sites to help attach covering material (3) to it, where the location of the hooks can be at the tip (4) of the protrusion (2) or somewhere along the length (5) of the protrusion (2). Additional adhesive material (6) can be impregnated or added to help better attach the covering material (3).

FIG. 17 illustrates an embodiment of the present invention and exemplifies an implantable device showing a zoomed-up illustration of a protrusion (2) and a part of the skeleton (1) of the implantable device, where the protrusion (2) is covered partially (3) or impregnated or covered diffusely (4) to help attach covering material (5) to it, and the location of said covering can be at the tip (3) of the protrusion (2) or anywhere along the length (4) of the protrusion (2).

FIG. 18 illustrates an embodiment of the present invention and exemplifies an implantable device showing protrusions (3) and the skeleton (2) of the implantable device that is put around an inflatable balloon (1) for delivery where the inflatable balloon (1) is used to expand and/or deploy the implantable device (such as transcatheter aortic valve replacement (TAVR)) comprising its associated protrusions (3) and covering material (4).

FIG. 19 illustrates an embodiment of the present invention and exemplifies an implantable device showing: in panel (A) the skeleton (1) of the implantable device, the skeleton (1) comprising components that can have an independent motion (from the rest of the skeleton), where said component can either be an entire part (such as a wire) (2) of the skeleton, or include specific segments (4) connected by connections (3) within the skeleton (1). These wires (2), or segments (4) that are connected by said connections (3) have more independent motion (folding and/or unfolding) unlike other wires, or components of the skeleton (1) linked together with specific connections. In panel (B), the illustration exemplifies another embodiment of the implantable device of the present invention where the skeleton (1) of the implantable device comprises more flexible wires (2) or segments (4) of more flexible wires, that can independently unfold into gaps around the implantable device, at least partially due to the paucity or lack of connections (3) between such segments of wires, where the connections (3) otherwise limit their independent motion. These flexible segments may sometimes be constrained (5) when there is no gap adjacent to them.

FIG. 20 illustrates an embodiment of the present invention and exemplifies an implantable device showing the skeleton (1) of the implantable device with exemplary protrusions (2), (3), (4), and (5) over the body of the implantable device. Some of the exemplary protrusions (2) are attached to one of the components, parts, or wires of the skeleton (1) of the implantable device. Some of the exemplary protrusions are attached to either two (3) or more (4) wires or components of the skeleton (1) of the implantable device. Some of the protrusions (5) are attached to the body of the implantable device itself.

FIG. 21 illustrates an embodiment of the present invention and exemplifies an implantable device showing a top view of an implantable device with its skeleton (1), protrusions (2), and covering material. The protrusions (2) are shaped in a loop-like form with two or more attachments to the body or the skeleton of the implantable device. The covering material can be attached to only one protrusion or protrusion loop at a time (3), or it can extend to cover multiple protrusions (7). The height of each loop (5), the width of each loop (6), and the distance (4) between the protrusions are fixed or variable, and they follow several patterns, for example, long then short (8) pattern, or long, medium and then short loop (9) pattern.

FIG. 22 illustrates an embodiment of the present invention and exemplifies an implantable device showing in panel (A) the skeleton (1) of the implantable device, protrusions (2), and covering material (3). The protrusions (2) form a loop that is attached to two wires or components of the skeleton (1) of the implantable device. The protrusions (2) are attached to the covering material (3) using mechanical attachment sites (4) such as hooks or sutures, or using adhesive material, etc. In panel (B) another embodiment of the implantable device is exemplified showing multiple protrusions (2), (5), and (6) attached to the skeleton (1) of the implantable device, and a covering material (3), where the protrusions (2), (5), and (6) are attached to the covering material (3) using mechanical attachment sites (4) such as hooks or adhesive material as the points of attachment for the covering material.

FIG. 23 illustrates an embodiment of the present invention and exemplifies an implantable device showing the skeleton (1) of the implantable device, and some of the protrusions (2) and (5), where the protrusions (2) and (5) are curved or shaped (3) to stack better (2) and (5) around a specific segment (4) of the implantable device that is curved when the implantable device is compressed inside a delivery catheter. Other protrusions (6), (7), and (8) are curved or shaped so that they can stack better against each other (6), (7), and (8) when the implantable device is compressed inside the delivery catheter.

FIG. 24 illustrates an embodiment of the present invention and exemplifies an implantable device showing the skeleton (1) of the implantable device, and protrusions (2). The protrusions can be attached to the covering material (7) using one or more mechanical attachment sites (3), (4), and (5) such as hooks or adhesive material, etc. These attachment sites can be at the base of the protrusion (5), the tip (3), or anywhere along the protrusion length (4). The distance between these protrusions (6) can be variable or constant.

FIG. 25 illustrates an embodiment of the present invention and exemplifies an implantable device showing a top view in panel (A) of the skeleton (1) of the implantable device, protrusions (2), and covering material (3), which is attached to the protrusions. The covering material (3) can be made from one piece that extends over many and/or all the protrusions (4) attached over the skeleton (1) of the implantable device using one or more mechanical attachment sites and/or sutures (7). The covering material (3) can also extend over the body (5) of the implantable device itself and be attached to the skeleton (1) of the implantable device using one or more mechanical attachment sites and/or sutures (6). In panel (B), another embodiment of the implantable device of the present invention is exemplified showing the skeleton (1) of the implantable device with a covering material (3) attached to multiple and/or all of the protrusions (2). Thus, in this embodiment, the covering material along with the protrusions on the skeleton of the implantable device form a sealing halo around the implantable device. In panel (C), another embodiment of the implantable device of the present invention is exemplified by showing the skeleton of the implantable device, where the skeleton is either covered (1) with a covering material (5) attached to multiple and/or all of the protrusions (3) or the skeleton is uncovered (4). Thus, this embodiment provides a partial covering of the skeleton of the implantable device that forms a distinct form of a sealing halo around a partial skeleton (1) part of the implantable device. Further, the aforesaid sealing halo cover is distinct and separate from another covering material (2) that is used to cover the part of the body of the implantable device itself. In panel (D), another embodiment of the implantable device of the present invention is exemplified by showing the skeleton (1) of the implantable device is partially covered along with part of the body of the implantable device with the same covering material (3) rather than distinct and separate covering materials of the former embodiment, and the same covering material of this embodiment can be attached to parts or whole of both the skeleton and body of the implantable device itself along with the protrusions (2) on them, and thus it can be used to cover the entire device (1).

FIG. 26 illustrates an embodiment of the present invention and exemplifies an implantable device showing the skeleton (1) of the implantable device, and protrusions (2), where the protrusions are attached to a covering material (3). The covering material (3) contains a foam-like material (4) that can help further cover the gaps surrounding the implantable device.

FIG. 27 illustrates an embodiment of the present invention and exemplifies in panel (A) an implantable device embodiment, where the protrusions (1) are attached to a flexible ring (3) which is built into the implantable device itself. In panel (B), an embodiment of the present invention exemplified a separate sealing halo unit comprising the protrusions (1) and the flexible ring (3) both covered by a covering material (2) (top ring-like structure), where a combination of all of these (1), (2), and (3) is referred to as the sealing halo unit that helps seal gaps around the implantable device, and it is used to be fused to the skeleton (4) of the implantable device (bottom ring-like structure). Here, the sealing halo unit can be either attached to or built into the implantable device such as a transcatheter aortic valve replacement (TAVR) or left atrial appendage (LAA) occluder (4). In panel (C), another embodiment of the implantable device of the present invention is exemplified, where the protrusions (1) are attached to a flexible ring (3) which is attached to the implantable device, and one or more covering material (2) is attached to these protrusions (1) and/or the ring (3).

FIG. 28 illustrates an embodiment of the present invention and exemplifies in panel (A), an implantable medical device, where the protrusions, covering material and/or sealing halo unit (1) can be refolded back in the same direction as the original compressed state before deployment. In the implantable device, the skeleton (3) of the implantable device and covering (2) of the implantable device are also displayed along with a pulling rod mechanism (4) which is used to pull all these components back into the delivery catheter. In panel (B), another embodiment of the implantable medical device of the present invention is shown with protrusions, covering material and/or sealing halo unit (1) that can be refolded back in the opposite direction as the original compressed state shown in panel (A). In panel (C), another embodiment of the implantable medical device of the present invention is shown with protrusions (1) attached to the skeleton (2) of the implantable device. The pulling rod mechanism (3) is also attached to the skeleton (2) of the implantable device. It can help in pulling the entire implantable device and its associated sealing halo unit (and/or protrusions and attached covering material) back into the delivery catheter as well as it helps in advancing them into position during delivery.

FIG. 29 illustrates an embodiment of the present invention and exemplifies in panel (A), an implantable medical device, where the protrusions (1) can swivel sideways (2) when they unfold when compared to the implantable device and/or its skeleton (3). In panel (B), another embodiment of the implantable medical device of the present invention is shown with protrusions (1) that can swivel parallel (2) when they unfold when compared to the implantable device and/or its skeleton (3).

FIG. 30 illustrates an embodiment of the present invention and exemplifies in panels (A) and (B) respectively, the top and side views of an implantable device where protrusions (4) and/or sealing halo unit (3) are attached to the outer aspect of the annulus (2) of the implantable device which here is a transcatheter aortic valve replacement (TAVR) (1).

FIG. 31 illustrates an embodiment of the present invention and exemplifies an implantable device showing the skeleton (1) of the implantable device, along with protrusions (2) that have independent motion from the skeleton (1) of the implantable device. Interconnections or links can be made or built between the protrusions (2) and can be attached anywhere along the length of the protrusions whether the tips (3) of the protrusions, or the body (4) of the protrusions.

FIG. 32 illustrates an embodiment of the present invention and exemplifies an implantable device showing a magnified illustration of an implantable device with protrusions (1) that may have an additional extension (2) such as an arch extension to enable better and smoother expansion. These extensions (2) can be made from memory metal or other flexible materials, these extensions (2) can also be attached to a covering material (4) along the skeleton (3) of the implantable device.

FIG. 33 illustrates an embodiment of the present invention and exemplifies an implantable device showing a sealing halo (1), where the protrusions (3) can be attached to a flexible stent-like ring (4), which can be attached to an implantable device, and a covering material (2) can also be attached to these protrusions (3) and/or the stent-like ring (4). A combination of all of these (1), (2), (3), (4) is another embodiment of a sealing halo unit of the present invention that helps seal gaps around the implantable device, such as a left atrial appendage (LAA) occluder, transcatheter aortic valve replacement (TAVR), etc. The sealing halo unit can be attached to or built into the implantable device, such as a TAVR, LAA occluder, or any other implantable medical device. The sealing halo unit can also be fused to the skeleton of the implantable device, such as an LAA occluder, TAVR, or any other implantable medical device.

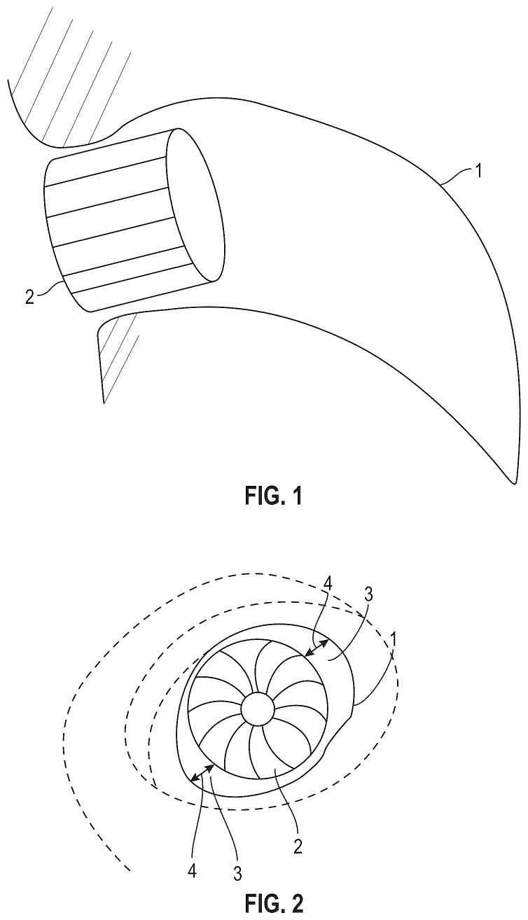

### DETAILED DESCRIPTION OF THE INVENTION

Detailed embodiments of the present invention are disclosed herein. However, it is to be understood that the disclosed embodiments are merely exemplary of the present invention, which may be embodied in various systems. Therefore, specific details disclosed herein are not to be interpreted as limiting, but rather as a basis for teaching one skilled in the art to variously practice the present invention.

All illustrations of the drawings are to describe selected versions of the present invention and are not intended to limit the scope of the present invention.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

Although any methods and materials similar or equivalent to those described herein can be used in the practice of the present invention, the exemplary methods, devices, and materials are described herein. For the present disclosure, the following terms are defined below. Additional definitions are set forth throughout this disclosure.

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a microbe, a microbial formulation, a pharmaceutical composition, and/or a method that “comprises” a list of elements (e.g., components, features, or steps) is not necessarily limited to only those elements (or components or steps), but may include other elements (or components or steps) not expressly listed or inherent to the microbe, microbial formulation, pharmaceutical composition and/or method. Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, the term “and/or” when used in a list of two or more items, means that any one of the listed items can be employed by itself or in combination with any one or more of the listed items. For example, the expression “A and/or B” is intended to mean either or both of A and B, i.e., A alone, B alone or A and B in combination. The expression “A, B and/or C” is intended to mean A alone, B alone, C alone, A and B in combination, A and C in combination, B and C in combination or A, B, and C in combination.

As used herein, the term “about” refers to a rough estimate of the number or amount of the quantity referred to and is in the vicinity of the actual number or figure immediately following said term, where the actual number or figure or amount could be slightly higher or lower.

As discussed above, there remains a need in the art for better implantable devices that when deployed in the requisite place in the body in case of a medical device or otherwise for a non-medical device, do not leave gaps and fully occlude the aperture or the cavity being targeted by said device.

Accordingly, in some embodiments of the present invention, it describes a new way to enable a better sealing and/or anchoring of various types of implantable medical devices such as LAA occluders, Transcatheter Aortic Valve Replacement (TAVR), Transcutaneous Mitral Valve Replacement, Atrial Septal Occluders, Patent Foramen Ovale Occluders, vascular plugs, etc. In other embodiments of the present invention, it provides implantable devices that can be used to build better occluder devices that can be used in non-medical devices and applications, for example, to occlude a tube, channel, conduit, and duct, such as a leaking oil pipe or water pipe, etc.

An embodiment of the present invention provides an implantable device as disclosed herein, the device comprising a main body to achieve a certain function such as occluding an abnormal cavity, aperture, or vessel; a valve to direct blood flow in a certain direction, wherein the main body of the implantable device has an underlying skeleton to support the main body of the device and to create and maintain its shape.

The present invention utilizes one or more protrusions/extensions with (or without) additional attached or linked covering material. Covering material can be attached to the protrusions and/or the body or the skeleton of the implantable device or any particular part of the implantable device. These protrusions and covering materials can be attached to, or built as a part of the body of the implantable device or its skeleton, or attached to any part of the implantable device. These protrusions and covering material when combined with an attachment ring can form a unit called a sealing halo unit.

Any one or more of these protrusions can have independent unfolding motion (from the implantable device and/or its skeleton), so any one or any combination of protrusions can extend into any gap on the outside or the periphery of the implantable device. Then the protrusions can form a scaffolding to support the covering material, that is attached to the protrusions and/or supported by them, thus the covering material can also unfold (and/or be lifted) by the protrusions to further seal these gaps around the implantable device. Length of protrusions is usually designed based on the expected of width of the gap around the implantable device. Because of the independent motion of the protrusions from the device itself, the protrusions will only unfold if there is a gap adjacent to any or a group or all of the protrusions, otherwise, the protrusion(s) will stay partially or completely folded.

In an embodiment of the present invention, it provides an implantable device, the implantable device comprising: a main body; a skeleton; one or more protrusions; and a covering material, wherein the main body is selected between an occluder and a prosthetic valve, wherein the occluder is selected from a group comprising occluders to occlude a cavity, aperture, vessel, tube, channel, conduit, and duct; wherein the prosthetic valve is a valve that directs and regulates liquid flow in a certain direction, and wherein the skeleton supports the main body of the implantable device and creates and maintains its shape.

In an embodiment of the present invention, it provides an implantable device for occluding an aperture or cavity in the body of an animal, wherein the device is a medical device, wherein the animal is a mammal. In another embodiment of the present invention as disclosed herein, wherein the mammal is human.

In an embodiment of the present invention, it provides an implantable device, wherein the implantable device is a medical device, wherein the medical device is an occluder for occluding a cavity, aperture, or vessel in the body of an animal, wherein the animal is a mammal. In another embodiment of the present invention, wherein the implantable device is a medical device, wherein the medical device enables better sealing and anchoring of implantable medical devices selected from a group of devices comprising left atrial appendage (LAA) occluders, Transcatheter Aortic Valve Replacement (TAVR), Transcutaneous Mitral Valve Replacement (TMVR), Transcatheter Tricuspid Valve Replacement (TTVR), Transcatheter Pulmonic Valve Replacement (TPVR), Atrial Septal Occluders, Patent Foramen Ovale Occluders, and vascular plugs.

In an embodiment of the present invention, it provides an implantable device as a replacement for a native valve in the body of an animal, wherein the device is a medical device, wherein the medical device is a prosthetic valve, wherein the animal is a mammal. In another embodiment of the present invention as disclosed herein, wherein the mammal is human.

In an embodiment of the present invention, it provides an implantable device, wherein the implantable device is a medical device, wherein the medical device is a prosthetic valve, wherein the prosthetic valve is a replacement for a native valve in the body of an animal as an artificial valve, wherein the animal is a mammal.

In an embodiment of the present invention, it provides an implantable device for occluding an aperture or cavity in an apparatus, wherein the apparatus comprises a leaking oil pipe or water piper, and wherein the device is a non-medical device.

In an embodiment of the present invention, it provides an implantable device, wherein the implantable device is a non-medical device, wherein the non-medical device is an occluder for occluding a tube, channel, conduit, or duct.

In an embodiment of the present invention, it provides an implantable device, wherein the implantable device is a non-medical device, wherein the medical device is a prosthetic valve, wherein the prosthetic valve is a replacement for a faulty valve in a tube, channel, conduit, or duct that regulates the flow of a liquid through said tube, channel, conduit, or duct.

In an embodiment of the present invention, it provides an implantable device comprising a main body, a skeleton, and a biocompatible covering material, wherein the implantable device is an LAA occluder. In another embodiment of the LAA occluder as disclosed herein, wherein the skeleton comprises a memory metal, the memory metal selected from a group comprising Nitinol, shape-memory alloys including copper-aluminum-nickel and nickel-titanium (NiTi), and other alloys containing a metal selected from a group comprising zinc, copper, gold, and iron. In another embodiment of the LAA occluder as disclosed herein, wherein the skeleton comprises other flexible materials. In another embodiment of the LAA occluder as disclosed herein, wherein the skeleton and the biocompatible covering material are linked or interlinked together to allow the main body and the biocompatible covering material to expand together as a unit with limited independent movement of the LAA occluder.

In an embodiment of the present invention, it provides an implantable device for the treatment of atrial fibrillation, the implantable device comprising a main body; a skeleton; and a covering material, wherein the implantable device for the treatment of atrial fibrillation is a left atrial appendage (LAA) occluder, wherein the covering material is a biocompatible covering material, and wherein the skeleton and the covering material support the main body of the implantable device and create and maintain its shape. In another embodiment of the present invention, wherein the skeleton comprises a memory metal, wherein the memory metal is selected from a group comprising Nitinol, shape-memory alloys, and other flexible materials, and wherein the shape-memory alloys include copper-aluminum-nickel and nickel-titanium (NiTi), and other alloys containing a metal selected from a group comprising zinc, copper, gold, and iron. In another embodiment of the present invention, wherein the skeleton and the biocompatible covering material are linked together to allow the main body and the biocompatible covering material to expand together as a unit with limited independent movement of the LAA occluder.

In an embodiment of the present invention, it provides an implantable device comprising a main body, an annulus, valve leaflets, a skeleton, and a biocompatible covering material, wherein the implantable device is a Transcatheter Aortic Valve Replacement (TAVR), a Transcatheter Mitral Valve Replacement (TMVR), Transcatheter Tricuspid Valve Replacement (TTVR), Transcatheter Pulmonic Valve Replacement (TPVR), wherein the implantable device is a prosthetic valve that directs and regulates liquid flow in a certain direction and it is a replacement for a native valve in the body of an animal as an artificial valve. In another embodiment of the implantable device as disclosed herein, wherein the skeleton comprises a memory metal, the memory metal selected from a group comprising of Nitinol, shape-memory alloys including copper-aluminum-nickel and nickel-titanium (NiTi), and other alloys containing a metal selected from a group comprising zinc, copper, gold, and iron. In another embodiment of the implantable device as disclosed herein, wherein the skeleton comprises other flexible materials. In another embodiment of the implantable device as disclosed herein, wherein the skeleton and the biocompatible covering material are linked together to allow the main body and the biocompatible covering material to expand as a unit with limited independent movement of the TAVR (or TMVR, TTVR, TPVR).

In an embodiment of the present invention, it provides an implantable device for the treatment of an abnormal valve in the body of an animal, the implantable device comprising: a main body; a skeleton; a covering material; an annulus; and valve leaflets, wherein the implantable device is a prosthetic valve that directs and regulates liquid flow in a certain direction and it is a replacement for a native valve in the body of an animal as an artificial valve, wherein the implantable device is a Transcatheter Aortic Valve Replacement (TAVR), a Transcatheter Mitral Valve Replacement (TMVR), Transcatheter Tricuspid Valve Replacement (TTVR), Transcatheter Pulmonic Valve Replacement (TPVR), wherein the animal is a mammal, wherein the covering material is a biocompatible covering material, and wherein the skeleton and the covering material support the main body of the implantable device and create and maintain its shape. In another embodiment of the implantable device as disclosed herein, wherein the skeleton comprises a memory metal, wherein the memory metal is selected from a group comprising Nitinol, shape-memory alloys, and other flexible materials, and wherein the shape-memory alloys include copper-aluminum-nickel and nickel-titanium (NiTi), and other alloys containing a metal selected from a group comprising zinc, copper, gold, and iron. In another embodiment of the implantable device as disclosed herein, wherein the skeleton and the biocompatible covering material are linked together to allow the main body and the biocompatible covering material to expand together as a unit with limited independent movement of the TAVR, TMVR, TTVR, or TPVR.

The present invention further discloses embodiments where the implantable device as disclosed herein can be used to modify an existing or new implantable device's design to obtain a modified device to help said modified device achieve better sealing and/or better anchoring of the device to the specific surface of aperture or cavity. This modification can be built as an add-on apparatus (referred to as a sealing halo unit) that can be fused or attached to the implantable device. In another embodiment of the present invention, the sealing halo device can be built into the design of a new or existing implantable device, to achieve better sealing and/or better anchoring.

In an embodiment of the present invention, it provides an implantable device to modify another implantable device, the implantable device comprising: a sealing halo unit, wherein the sealing halo unit is selected from a group of apparatus to be fused or attached to the implantable device, wherein the sealing halo unit is selected from a group comprising a built-in and an add-on apparatus to modify the another implantable device, wherein the another implantable device is an existing or new implantable device, wherein the implantable device modifies another implantable device leading to better sealing and/or better anchoring of the another implantable device to the specific surface of the aperture or cavity in the body of an animal, and wherein the animal is a mammal.

In an embodiment of the present invention, it provides a flexible halo, which is a sealing halo unit that is fused or attached to, or built as a part of the implantable device as disclosed here in the present invention, around the outside of the device. This halo may comprise covering material that is supported by independent flexible protrusions. The components of the halo are usually independent, and they usually only expand into gaps around the device that are not filled by the body of the device itself, thus providing better sealing.

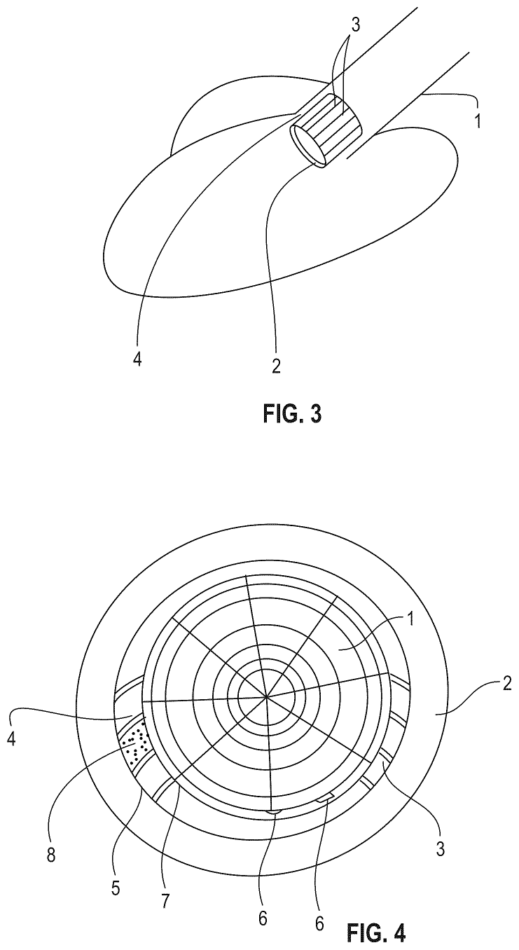

In an embodiment of the present invention, it provides an implantable device comprising a main body and a skeleton, wherein one or more flexible or semi-flexible independent, semi-independent and/or linked protrusions or extensions are attached to, or built-in as a part of the main body and/or skeleton of the implantable device. These protrusions can unfold or expand into the outside or the periphery of the implantable device, typically into spaces around the device that are not filled by the main body of the implantable device. Thus, the protrusions can help fill these gap(s) between the implantable device and the inner surface of the target aperture, vessel or cavity, etc. as shown in FIG. 3. These protrusions can function as a scaffolding to support attached covering material, which can be made from a variety of flexible materials as shown in FIG. 3.

Covering material can be made using porous, semi-porous or non-porous membrane, mesh (or other flexible or nonflexible material) that can provide additional sealing as shown in FIG. 3. Protrusion(s) with or without the covering material would provide the flexibility and adaptability to implantable devices, so they can better seal the spaces between them and the target aperture, cavity, vessel etc. and it also can provide better anchoring and stability to the implantable device, so that it is less likely to embolize, or move from its location as shown in FIG. 3.

In an embodiment of the present invention, it provides a modified implantable device comprising a main body and a skeleton, wherein the modified implantable device is obtained by modifying an existing implantable device, wherein the implantable device is a LAA occluder device, wherein one or more flexible or semi-flexible independent, semi-independent and/or linked protrusions or extensions are attached to the periphery of the modified implantable device, wherein the protrusions can independently unfold into the gaps between the inner surface of the LAA cavity and the LAA occluder itself. These protrusions, individually or in any combination, can function as scaffolding, thus lifting attached covering material, thus covering any gaps between the LAA occluder and the LAA walls, and providing additional sealing of these gaps as shown in FIG. 3. These protrusions will usually only unfold into gaps or free spaces that are not already occluded by the body of the implantable device itself.

In an embodiment of the present invention, it provides an implantable device, the implantable device comprises: a main body; a skeleton; one or more protrusions; and a covering material, wherein the main body is selected from a group comprising an occluder and a prosthetic valve, wherein the occluder is selected from a group comprising occluders to occlude a cavity, aperture, vessel, tube, channel, conduit, and duct; wherein the prosthetic valve is a valve that directs and regulates liquid flow in a certain direction, wherein the skeleton and the covering material support the main body of the implantable device and create and maintain its shape, wherein the covering material is attached to the implantable device, wherein the covering material is a biocompatible covering material, wherein the one or more protrusions are attached or linked to the covering material, and wherein the one or more protrusions, and the covering material, expand into gaps around the implantable device to fill in the gaps not filled by the main body and skeleton of the implantable device. In another embodiment of the implantable device as disclosed herein, wherein the implantable device is a medical device, wherein the medical device is an occluder for occluding a cavity, aperture, or vessel in the body of an animal, wherein the animal is a mammal. In another embodiment of the implantable device as disclosed herein, wherein the implantable device is a medical device, wherein the medical device is a prosthetic valve, wherein the prosthetic valve is a replacement for a native valve in the body of an animal as an artificial valve, wherein the animal is a mammal. In another embodiment of the implantable device as disclosed herein, wherein the implantable device is a non-medical device, wherein the non-medical device is an occluder for occluding a tube, channel, conduit, or duct. In another embodiment of the implantable device as disclosed herein, wherein the implantable device is a non-medical device, wherein the medical device is a prosthetic valve, wherein the prosthetic valve is a replacement for a faulty valve in a tube, channel, conduit, or duct that regulates the flow of a liquid through said tube, channel, conduit, or duct. In another embodiment of the implantable device as disclosed herein, wherein the implantable device is a medical device, wherein the medical device enables better sealing and anchoring of implantable medical devices selected from a group of devices comprising left atrial appendage (LAA) occluders, Transcatheter Aortic Valve Replacement (TAVR), Transcutaneous Mitral Valve Replacement (TMVR), Transcatheter Tricuspid Valve Replacement (TTVR), Transcatheter Pulmonic Valve Replacement (TPVR), Atrial Septal Occluders, Patent Foramen Ovale Occluders, and vascular plugs. In another embodiment of the implantable device as disclosed herein, wherein the one or more protrusions are selected from a group comprising protrusions that are flexible and independent, semi-flexible and independent, semi-independent, and linked and built-in as part of the main body and/or skeleton of the implantable device. In another embodiment of the implantable device as disclosed herein, wherein the covering material is selected from a group of materials comprising porous, semi-porous, or non-porous membranes, a mesh made of porous, semi-porous, or non-porous materials, and other flexible or nonflexible materials that can provide additional sealing to the implantable device.

In an embodiment of the present invention, it provides an implantable device for the treatment of atrial fibrillation, the implantable device comprises: a main body; a skeleton; one or more protrusions; and a covering material, wherein the implantable device for the treatment of atrial fibrillation is a left atrial appendage (LAA) occluder, wherein the skeleton and the covering material support the main body of the implantable device and create and maintain its shape, wherein the covering material is attached to the implantable device, wherein the covering material is a biocompatible covering material, wherein the one or more protrusions are attached or linked to the covering material, and wherein the one or more protrusions, and the covering material, expand into gaps around the implantable device to fill in the gaps not filled by the main body and skeleton of the implantable device. In another embodiment of the implantable device for the treatment of atrial fibrillation as disclosed herein, wherein the skeleton comprises a memory metal, wherein the memory metal is selected from a group comprising Nitinol, shape-memory alloys, and other flexible materials, and wherein the shape-memory alloys include copper-aluminum-nickel and nickel-titanium (NiTi), and other alloys containing a metal selected from a group comprising zinc, copper, gold, and iron. In another embodiment of the implantable device for the treatment of atrial fibrillation as disclosed herein, wherein the skeleton and the biocompatible covering material are linked together to allow the main body and the biocompatible covering material to expand together as a unit with limited independent movement of the LAA occluder. In another embodiment of the implantable device for the treatment of atrial fibrillation as disclosed herein, wherein the one or more protrusions are selected from a group comprising protrusions that are flexible and independent, semi-flexible and independent, semi-independent, and linked and built-in as part of the main body and/or skeleton of the implantable device. In another embodiment of the implantable device for the treatment of atrial fibrillation as disclosed herein, wherein the covering material is selected from a group of materials comprising porous, semi-porous, or non-porous membranes, a mesh made of porous, semi-porous, or non-porous materials, and other flexible or nonflexible materials that can provide additional sealing to the implantable device.

In an embodiment of the present invention, it provides an implantable device for the treatment of an abnormal valve in the body of an animal, the implantable device comprises: a main body; a skeleton; one or more protrusions; a covering material; an annulus; and valve leaflets, wherein the implantable device is a prosthetic valve that directs and regulates liquid flow in a certain direction and it is a replacement for a native valve in the body of an animal as an artificial valve, wherein the implantable device is a Transcatheter Aortic Valve Replacement (TAVR), a Transcatheter Mitral Valve Replacement (TMVR), Transcatheter Tricuspid Valve Replacement (TTVR), Transcatheter Pulmonic Valve Replacement (TPVR), wherein the animal is a mammal, wherein the skeleton and the covering material support the main body of the implantable device and create and maintain its shape, wherein the covering material is attached to the implantable device, wherein the covering material is a biocompatible covering material, wherein the one or more protrusions are attached or linked to the covering material, and wherein the one or more protrusions, and the covering material, expand into gaps around the implantable device to fill in the gaps not filled by the main body and skeleton of the implantable device. In another embodiment of the implantable device for the treatment of an abnormal valve in the body of an animal as disclosed herein, wherein the skeleton comprises a memory metal, wherein the memory metal is selected from a group comprising Nitinol, shape-memory alloys, and other flexible materials, and wherein the shape-memory alloys include copper-aluminum-nickel and nickel-titanium (NiTi), and other alloys containing a metal selected from a group comprising zinc, copper, gold, and iron. In another embodiment of the implantable device for the treatment of an abnormal valve in the body of an animal as disclosed herein, wherein the skeleton and the biocompatible covering material are linked together to allow the main body and the biocompatible covering material to expand together as a unit with limited independent movement of the TAVR, TMVR, TTVR, or TPVR. In another embodiment of the implantable device for the treatment of an abnormal valve in the body of an animal as disclosed herein, wherein the one or more protrusions are selected from a group comprising protrusions that are flexible and independent, semi-flexible and independent, semi-independent, and linked and built-in as part of the main body and/or skeleton of the implantable device. In another embodiment of the implantable device for the treatment of an abnormal valve in the body of an animal as disclosed herein, wherein the covering material is selected from a group of materials comprising porous, semi-porous, or non-porous membranes, a mesh made of porous, semi-porous, or non-porous materials, and other flexible or nonflexible materials that can provide additional sealing to the implantable device.

In an embodiment of the present invention, it provides a sealing halo unit, comprising: an attachment ring; one or more protrusions; and a covering material, wherein the one or more protrusions and the covering material are attached or built onto the attachment ring to provide the sealing halo unit, wherein the sealing halo unit provides better sealing and/or better anchoring of the implantable device to a specific surface, wherein the sealing halo unit is a flexible halo that is further fused to or attached to or built as part of an implantable device around the outside of the implantable device, and wherein the one or more protrusions, the covering material, and the attachment ring independently expand into gaps around the implantable device to fill in the gaps not filled by the main body and skeleton of the implantable device to provide better sealing and/or better anchoring. In another embodiment of the sealing halo unit as disclosed herein, wherein the one or more protrusions support the attached covering material to maintain its structure and are selected from a group comprising protrusions that are flexible and independent, semi-flexible and independent, semi-independent, and linked and built-in as part of attachment ring, and wherein the covering material is selected from a group of materials comprising porous, semi-porous, or non-porous membranes, a mesh made of porous, semi-porous, or non-porous materials, and other flexible or nonflexible materials that can provide additional sealing to the implantable device.

In an embodiment of the present invention, it provides an implantable medical device comprising a skeleton, a main body, one or more protrusions, a biocompatible covering membrane, and additional components including valve leaflets and a combination thereof. The various embodiments as disclosed hereinabove and through the examples provided hereinbelow address the problem and need to fill in the gaps that form between the deployed implantable device and the wall of the target organ, aperture, cavity, vessel, tube, conduit, duct, etc. whether for its medical or non-medical use. The implantable device can be deployed in a compressed or folded conformity or design and upon deployment and reaching its target site including a target organ, aperture, cavity, vessel, tube, conduit, duct, etc., it unfolds and takes on the shape as best as it can to occlude the said target site as desired and the remaining gaps are filled by the one or more protrusions and/or additional extensions along with the covering material to better seal and provide better adherence of the implantable device of the present invention to the target site.

In the various embodiments of the present invention, the one or more protrusions and/or other additional extensions as the case may be are attached to the main body or the skeleton of the implantable device as disclosed herein and can have independent motion to unfold into the gaps between the implantable device and the target organ wall. These protrusions and extensions do not unfold if there is no gap adjacent to them, so they stay compressed (folded) adjacent to the body of the implantable medical device. These protrusions are flexible or semi-flexible and have an independent motion, they unfold completely or partially depending on how much gap is present between the implantable device and the target organ wall. The covering material as disclosed herein is attached to or supported by the protrusions. The covering materials are attached to the protrusions and unfold partially or completely based on the unfolding of the protrusions.

The invention will be further explained by the following Examples, which are intended to be purely exemplary of the invention and should not be considered as limiting the invention in any way.

### EXAMPLES

The following example provides exemplary embodiments of the implantable devices and sealing halo units of the present invention.

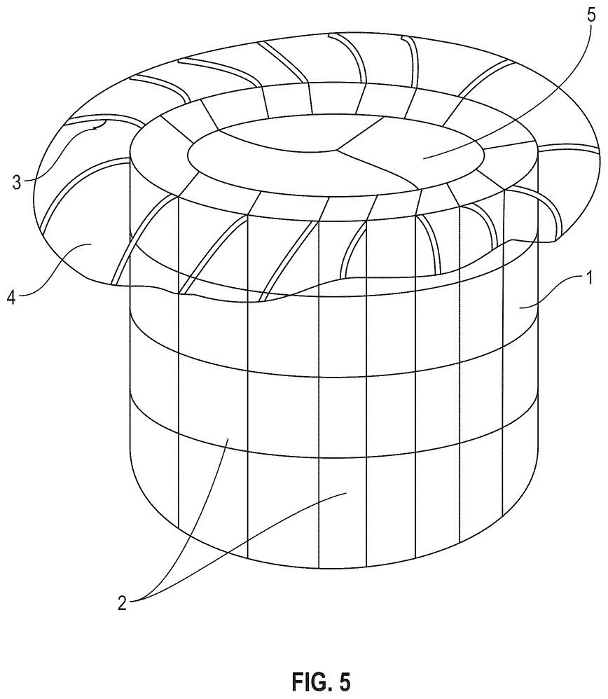

An exemplary implantable medical device of the present invention, as shown in FIG. 4 provides an implantable device where the protrusions can be used for scaffolding and/or to support other components such as covering materials, the covering materials selected from a group comprising biocompatible mesh, porous, semi-porous, and nanoporous membranes, and wherein the protrusions help move other components, that are part of the device or attached to the device, to further seal any gaps. FIG. 4 provides an example of the implantable medical device (1) that is deployed (i.e., unfolds when deployed) inside a target vessel, aperture, or cavity. As shown in the illustration, gaps (3) and (4) can form between the deployed implantable device (1) and the wall of the target organ (2) and (5). Protrusions (shown as parallel lines when unfolded and lumps/flanges when compressed) are attached to the body of the implantable medical device or its skeleton. Protrusions can have independent motion to unfold into the gaps (3) and (4) between the implantable device and the target organ wall. Protrusions do not unfold (as seen with unfolded lumps/flanges (6)) if there is no gap adjacent to them, so they stay compressed (folded) adjacent to the body of the implantable medical device. Because these protrusions are flexible or semi-flexible and possess an independent motion, they can either unfold completely (as seen in the gaps (3) and (4) represented by long parallel lines) or partially (as seen in the gap (4) represented by a shorter parallel line (7) at the end of the said gap (4)), depending on how much gap is present between the implantable device (1) and the target organ wall (2) and (5). Covering material (8) is attached to or supported by the protrusions. Covering materials are attached to or supported by the protrusions and unfold partially or completely based on the unfolding of the protrusions.

Another exemplary implantable medical device of the present invention, as shown in FIG. 5 provides another representative implantable device of the present invention where the implantable medical device body (1) is supported by a skeleton (2). Protrusions (3) are attached to the body or the skeleton of the medical device. Covering material (4) is attached to or supported by the protrusions, and the covering material can move with the protrusions. Here an example of the functional part of the implantable medical device (1) embodiment of the present invention is shown for a valve leaflet (5).

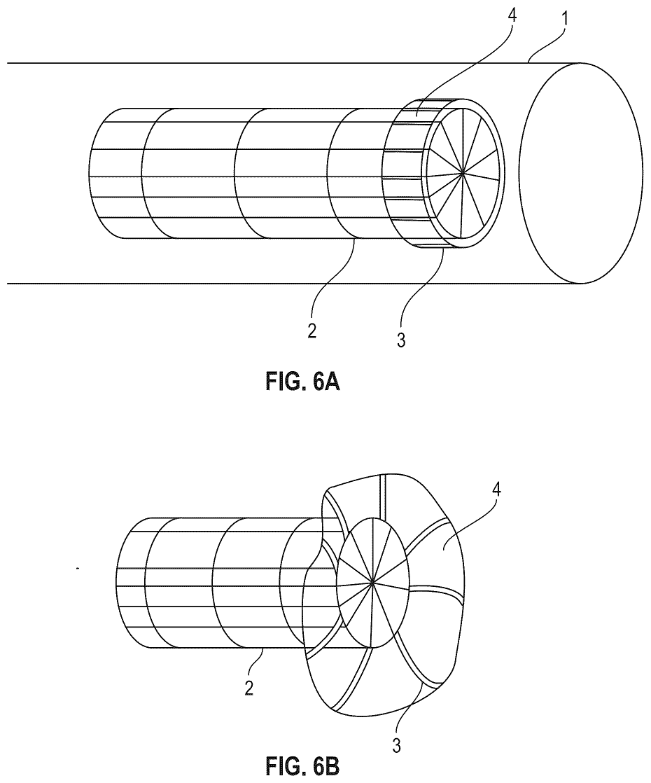

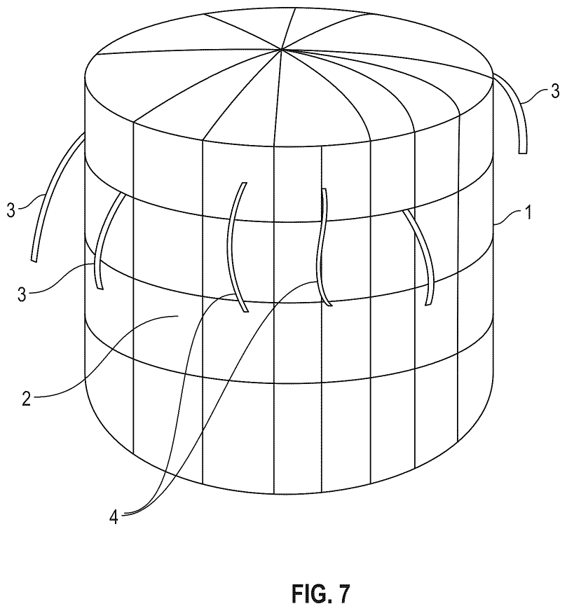

Another exemplary implantable medical device of the present invention, as shown in FIG. 6 provides a representative implantable device where the disclosed implantable medical device comprises protrusions and/or extensions that can be placed in a folded or compressed position alongside the implantable device itself that are attached to inside the delivery catheter. Then once the implantable device is released from the delivery catheter and it starts unfolding, the protrusions that are attached to the implantable devices or built as part of it can also unfold at the same time, before or shortly thereafter, typically based on the presence of a gap that is not filled by the implantable device itself as shown in FIG. 6A, and FIG. 6B. The present invention is exemplified in FIG. 6A by providing a representative mechanism by which the implantable device (2) of the present invention is delivered is as compressed inside a delivery catheter (1). The protrusions (3) and associated covering material (4) are folded or compressed alongside the compressed medical device (2). Further, in FIG. 6B the same implantable device (2) is shown after being released from the delivery catheter. The protrusions (3) will unfold if there is a sufficient gap(s) around the implantable device. Covering material (4) that is attached to the protrusions will also unfold whenever the protrusions that they are attached to move too.

Another exemplary implantable device of the present invention, as shown in FIG. 7 provides a representative implantable device where the protrusions or extensions can be built as a part of the implantable device structure and/or its skeleton itself, or they can be attached to any part of the implantable device components such as the skeleton, any interior or exterior part of the device, superior, inferior and/or the side of the device. FIG. 7 provides a representative implantable device where the protrusions can be attached to any part of the implantable device, including the periphery of the device, the inner part, top or bottom surface of the device, interior of the device, top or bottom surface or side of the skeleton, and/or all or any combination of these surfaces or parts. FIG. 7 demonstrates the representative implantable device's skeleton (1) along with the body (2) of the implantable device with protrusions (3) attached to specific locations on the skeleton (1) of the implantable device (1), and other protrusions (4) can also be attached to the body (2) of implantable devices. Further, another exemplary implantable device of the present invention provides that the protrusions can be made from the same material as the skeleton or the main body of the implantable device, said material selected from a group comprising Nitinol, other memory metals, and other flexible materials. They also can be built using different material(s) which may have different properties than the components of the implantable device itself.

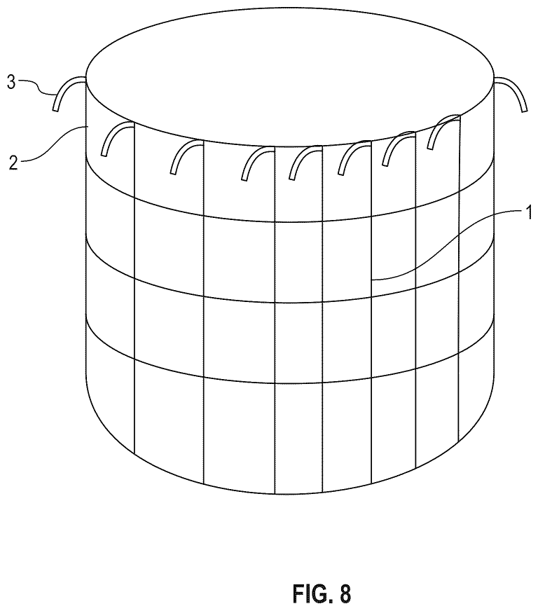

Another exemplary implantable device of the present invention, as shown in FIG. 8 provides a representative implantable device where the protrusions, all or any one of the protrusions can be an extension of the components (such as wires) that form the skeleton of the implantable device, other components of the implantable device body, the skeleton of the implantable device, or any other component of the skeleton of the implantable device. By being an extension of the implantable device or the skeleton, the protrusions can form an integral part of the implantable device, thus increasing the stability of the protrusions in relationship to the implantable device itself. In this case, the protrusions typically extend out partially or completely from the periphery of the implantable device when folded or unfolded as shown in FIG. 8. FIG. 8 demonstrates the representative implantable device showing that the protrusions (3) can be an extension of a wire (2) (or other components) of the skeleton (1) of the implantable device.

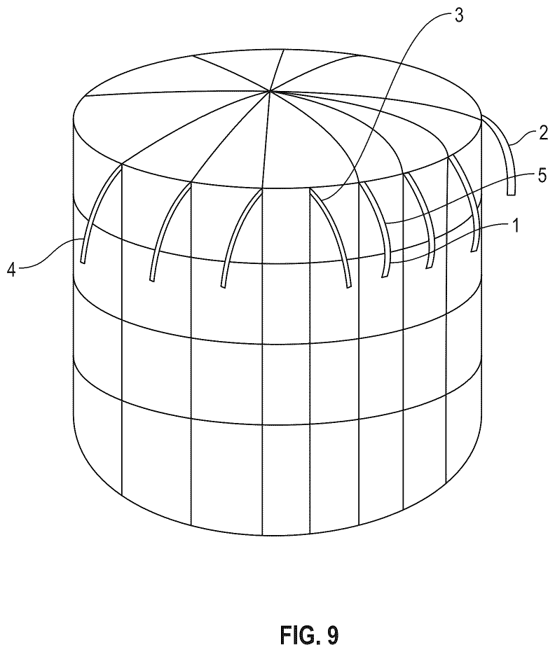

Another exemplary implantable medical device of the present invention, as shown in FIG. 9 provides a representative implantable device where the protrusions can have the same physical properties (such as length, thickness, width, etc.), composition, and/or mechanical properties as the other parts and/or components (or wires) used to build the skeleton of the device. The protrusions can also have different physical properties (being thinner, longer, shorter etc.), mechanical properties (more flexible, have more or less tensile strength, have different elasticity, have different malleability, have different hardness, have different ductility, have different creep and slip, have different deformity, have different resilience, or other mechanical properties), and/or composition that is/are different from the skeleton (or any or all of the skeleton's components) or the body of the implantable device itself and/or any other components of the implantable device itself. These features can be very helpful, for example, unfolding thinner protrusions at a lower force than the skeleton of the implantable device's body will minimize damage to the adjacent structures when the implantable device is deployed as shown in FIG. 9. FIG. 9 demonstrates the representative implantable device showing various forms and styles of protrusions (2), (3), (4), and (5) over the skeleton (1) of the implantable device, including thin protrusions (3), thicker protrusions (2), thicker and longer protrusions (4), and longer protrusions with variable thickness (5).

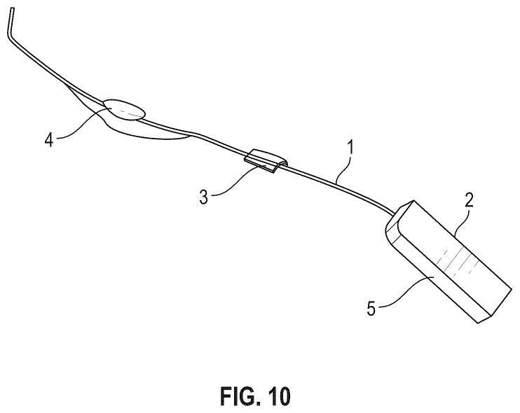

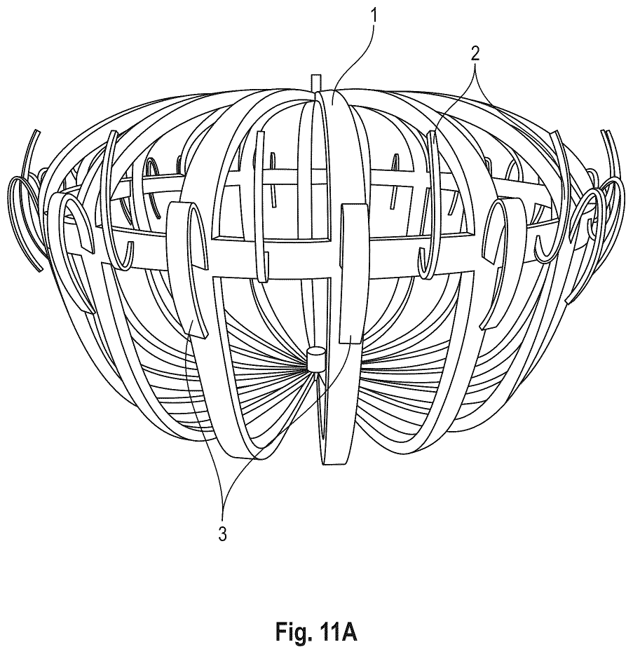

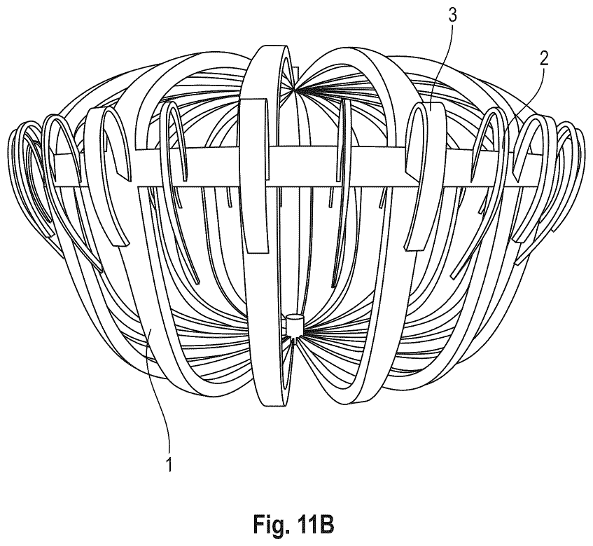

Further, the exemplary implantable device of the present invention, as shown in FIG. 9 above, provides a representative implantable device where the protrusions have different physical properties in terms of their width, thickness, length, composition, mechanical properties including flexibility, tensile strength, elasticity, malleability, hardness, ductility, creep and slip, deformity, resilience, or other mechanical properties. The aforementioned different physical properties can be between the various protrusions (refer to FIG. 9) or within the same protrusion (refer to FIG. 10). FIG. 10 demonstrates a representative singular protrusion showing the specific protrusion with protrusion (1) with variable thickness (5) and width (2), variable material composition (3), and combination of variable material composition and other physical properties such as width (4). Using different materials gives the protrusion desired mechanical properties that enable better unfolding, compression, etc. For example, using flexible material in building certain segments of the protrusion enables predetermined movement or flexing of the protrusion. These differences in physical and/or mechanical properties and/or composition between various protrusions, and also within any one protrusion can follow different patterns. For example, a long and thick protrusion can be located adjacent to a thin and short protrusion (on the implantable device), then a long thick protrusion next to a thin short protrusion as shown in FIG. 11.

Another exemplary implantable device of the present invention, as shown in FIG. 11 provides a representative implantable device where the protrusions can have uniform orientation or different orientations among themselves and/or in relation to the implantable device. Orientations of the protrusions can have a pattern, for example, one protrusion can be oriented downward in relationship to the body of the implantable device, then the next protrusion can be upward, and then this pattern can be repeated one and/or multiple times as can be seen in FIG. 11. FIG. 11A demonstrates a representative implantable device showing protrusions (2) and (3) attached to the skeleton (1) of the implantable device. The protrusions (2) have a similar orientation and their orientation alternates in pattern with another group of protrusions (3). The protrusions (2) have similar width and length, but they alternate in a pattern and orientation with the other group of protrusions (3). FIG. 11B demonstrates a representative implantable device showing the skeleton (1) of the implantable device, where protrusions (2) and (3) are attached to the implantable device with different lengths and widths in a pattern, for instance, a wide and short protrusion (3) followed by a narrow and longer protrusion (2) in a pattern in the same orientation.

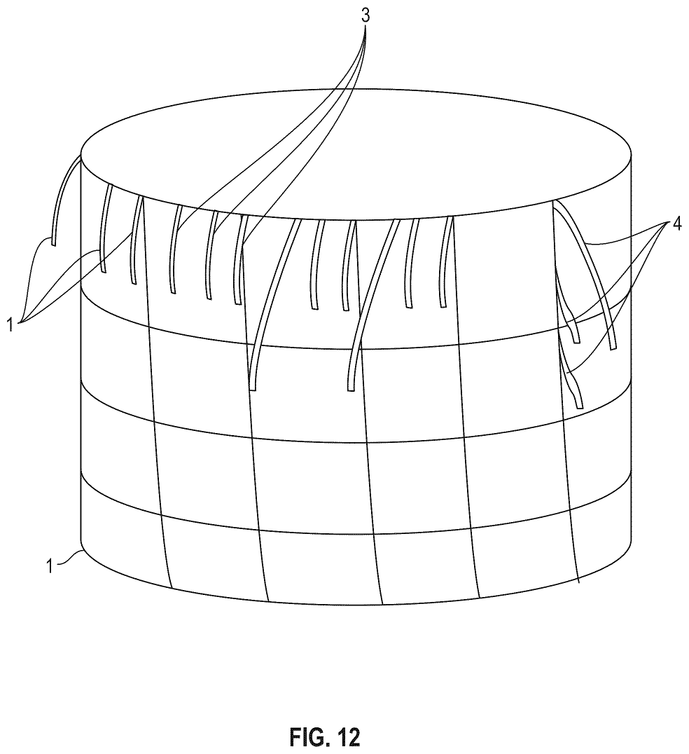

Another exemplary implantable device of the present invention, as shown in FIG. 12 provides a representative implantable device where the protrusions have the same length or different lengths in random or in a pattern to achieve a certain function. For example, a long protrusion, medium length protrusion, then a short protrusion followed by long, medium, and short protrusions in a pattern around the implantable device as seen in FIG. 12. FIG. 12 demonstrates a representative implantable device showing protrusions (2), (3), and (4) and the skeleton (1) of the implantable device. Various shapes or lengths of protrusions are attached to the skeleton (1) of the implantable device in various patterns including long, medium, and short lengths (2). This pattern can repeat itself one or more times (3). The protrusions can be attached in different patterns to the skeleton of the implantable device orientation and alternate in a pattern with other protrusion groups (3) and (4).

Another embodiment and example of the present invention provides the implantable medical device as disclosed herein, wherein the protrusions have variable lengths. In some embodiments and examples of the present invention providing the implantable medical device as disclosed herein, wherein the length variation can be random. However, the variability in protrusion lengths can have consistent and/or repeating patterns as exemplified in FIG. 12.

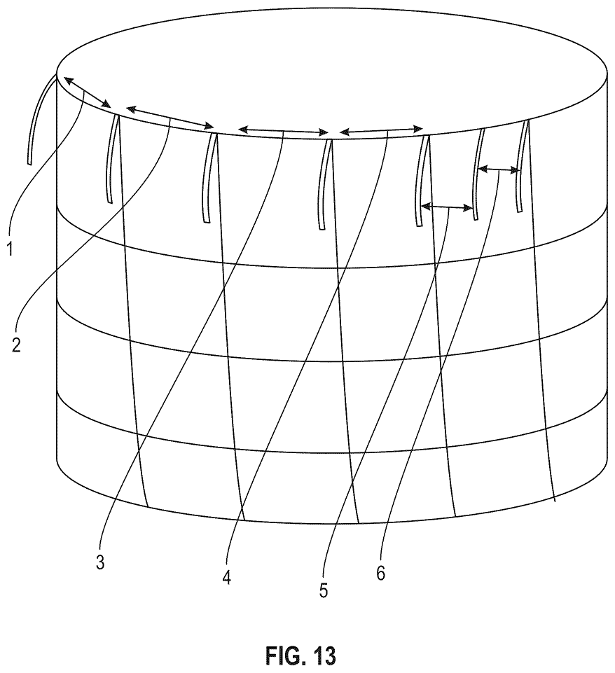

Another exemplary implantable device of the present invention, as shown in FIG. 13 provides a representative implantable device where the attachment sites of the protrusions to the implantable device can be chosen so that they are spaced vertically, diagonally, parallel to each other, or they can form various patterns to achieve different functions. FIG. 13 demonstrates a representative implantable device showing varying lengths of distance (1) to (6) between protrusion attachment sites on the skeleton of the implantable device. Exemplified distance between two protrusion attachment sites (1) can be the same as the distance between a separate set of two protrusions (4), or it can be different such as for different distances (2) and (3) between two other protrusion sets; or distance between the body of the protrusions (5) or various parts of the protrusions (6). Variations in distance between the attachment sites and/or the various parts of the unfolded or folded segments of the protrusions such as (1), (2) (3), (4), (5), and (6) can have a distinct pattern.

Having protrusions with variable lengths can enable a better sealing of any gap, whether, it is a narrow or wide gap. For example, in an example of the present invention of the implantable device, as disclosed herein, a short protrusion will extend and fill a narrower gap, while a longer protrusion will extend only into wider gaps, and stay folded if the gap is narrow.

Another example and embodiment of the present invention providing the implantable device as disclosed herein, wherein the distance between the protrusion's attachment sites to the implantable device and/or the distance between the protrusions themselves either folded or unfolded can be uniform or variable. These distances may also have a pattern. For example, a short distance (between the protrusions attachment site(s) to the skeleton or the body of the implantable device) is followed by a longer distance and so on as shown in FIG. 13.

In the various embodiments and examples of the present invention, in the disclosed implantable device, the protrusions can be curved, straight, or have other shapes. The protrusions' shapes can be uniform, or they can be variable in shape, whether randomly or in a pattern that may repeat itself.

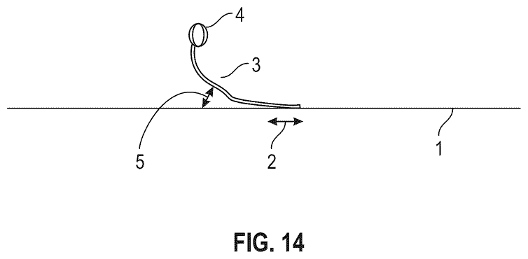

Another exemplary implantable device of the present invention, as shown in FIG. 14 provides a representative implantable device where the one or more protrusions are attached to the implantable device at an angle to the underlying skeleton and/or the body of the implantable device, wherein the angle can range from between 0° to 360°. FIG. 14 demonstrates a representative single protrusion on the skeleton of a representative implantable device showing a magnified illustration of an attachment site of a protrusion (3) attached to a part of the skeleton (1) of the implantable device of the present invention. The protrusion (3) can assume different shapes when folded or during unfolding. For example, in this illustration, it is curved, and it may have a hook (4) or other variations to enable the attachment of the covering material or to confer additional functions. The width (2) of the attachment site can be variable or patterned along with the angle (5) between the skeleton (1) of the implantable device and the protrusion (3). The angle can be present when the disclosed implantable device and the protrusions are compressed inside the delivery catheter, but it also can be present only when the device is deployed and the protrusion(s) unfolded. The angle can change and become variable based on the presence or absence of a gap around the implantable device (the angle is typically close to zero when the device is compressed inside the catheter before the implantable device is released). Different protrusions can have different angles to the underlying skeleton and/or the body of the implantable device, whether the protrusions are folded or unfolded.

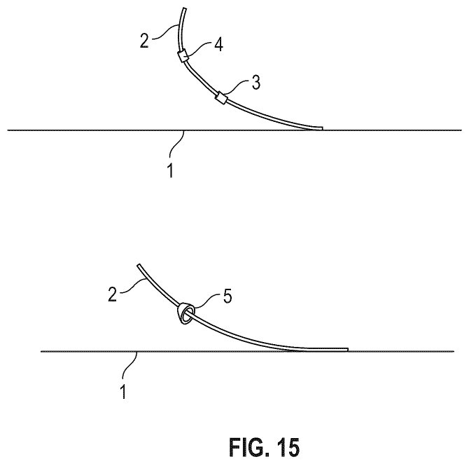

Another exemplary implantable device of the present invention, as shown in FIG. 15 provides a representative implantable device where the protrusions are usually made from flexible elements, which would allow it to bend, fold and unfold. But in some embodiments of the present invention, protrusions can have flexible joint(s) located somewhere along their length, to allow the protrusions to unfold to only the right length to occlude the gap around the implantable device, and to avoid excessive unfolding that may result in injury to the surrounding tissue. These flexible joints can be created by: making segment(s) within the protrusion from a different composition when compared to the other part of the protrusion, by using segment(s) of the protrusion with different physical properties than the other segments of the protrusion (for example, making a segment of the protrusion thinner and/or narrower than the other segments can make it more flexible), making segment(s) with different mechanical properties from the other segments of the protrusions, in addition to that, flexible segments can be made using articulating mechanism, as exemplified in FIG. 15. FIG. 15 demonstrates a representative single protrusion on the skeleton of a representative implantable device showing in the top panel a magnified illustration of a protrusion (2) attached to a part of the skeleton (1) of the implantable device. One or multiple flexible segments (3) and (4) within the protrusion (2) can be made from the same material as the rest of the protrusion (2), but they can be made more flexible because they have different physical properties such as being thinner, or narrower, etc., so they can bend more, or flex more, etc. These segments (3) and (4) can alternatively be made from other materials that are different from the rest of the protrusion (2), wherein these different materials give additional flexibility. In the bottom panel, another embodiment of the implantable device of the present invention is illustrated showing a protrusion (2) attached to a part of the skeleton (1) of the implantable device, the protrusion (2) comprising a mechanical joint (5) which allows additional flexibility and bending.

In another embodiment and example of the present invention providing the implantable medical device as disclosed herein, wherein the protrusions have articulating joints or one or more flexible segments at the attachment site to the implantable device itself, and/or anywhere along the length of the protrusions to enable the folding and unfolding of these protrusions. These flexible segments can consist of thinner or more malleable material due to different physical or mechanical properties than other segments of the protrusion to allow folding and unfolding, or may be built using a different and more flexible material to enable folding as exemplified in FIG. 15.

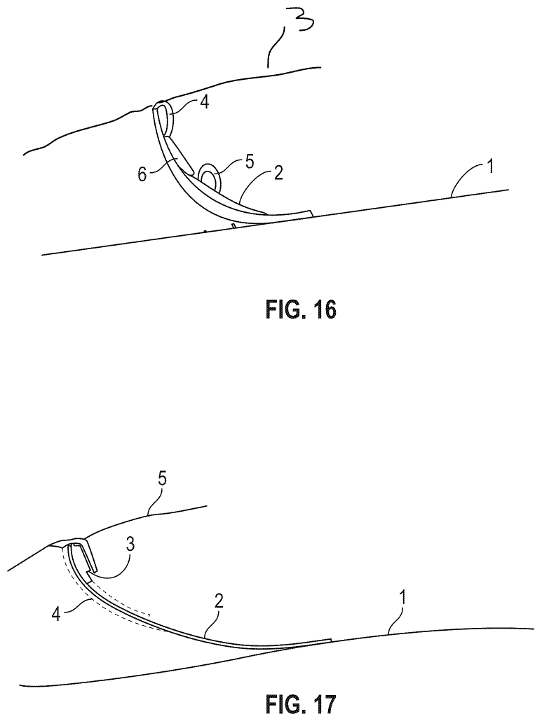

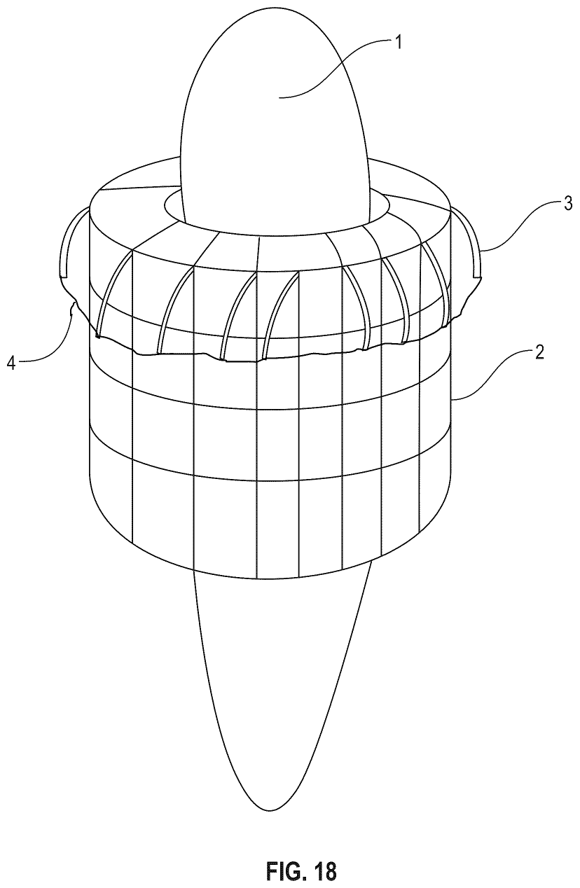

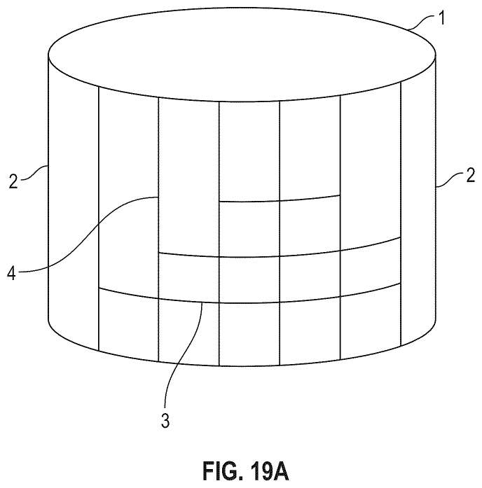

In another embodiment and example of the present invention providing the implantable medical device as disclosed herein, wherein the protrusions can be folded or compressed, said protrusions can be located parallel and/or closer to the main body of the implantable device, wherein the implantable device is inside the delivery catheter ready for deployment. But when the device is released, then the protrusion(s) will unfold only if there is a space or a gap that is not filled by the implantable device itself as exemplified in FIG. 16 and FIG. 17.

In some of the embodiments and examples of the present invention, the protrusions can be made from or can contain radio-opaque material, so they can be seen on X-ray or by fluoroscopy. In some of the embodiments and examples of the present invention, protrusions may also be made from the same material as the skeleton, but they also can be made from different material(s). In some of the embodiments and examples of the present invention, the material used for the protrusions can be metallic, or made from other materials selected from a group comprising plastic, and rubber. Preferably, in some of the embodiments and examples of the present invention, the material used for the protrusions is made from memory metal, preferably including Nitinol.

In some of the embodiments and examples of the present invention, in the implantable device as disclosed herein, the one or more protrusions have the same thickness, shape and/or width as the wires or other components of the main skeleton of the implantable device, but also can have different physical and/or mechanical properties from them.

In some of the embodiments and examples of the present invention, in the implantable device as disclosed herein, the protrusions can be advanced inside a catheter in a compressed form along with the implantable device inside the catheter, then any one or more of the protrusions can unfold partially or completely once the implantable device is released from the delivery catheter. The unfolding process is based on the memory properties of the protrusions' underlying material and also based on the presence or absence of gap(s) between the deployed implantable device and surrounding adjacent tissue.

Another exemplary implantable device of the present invention, as shown in FIG. 16 provides a representative implantable device where the one or more protrusions have built-in or attached hooks, one or more mechanical attachment sites, or links. These can help attach other materials such as membranes, mesh, etc., or other covering materials that are disclosed in the present invention. These attachments can be located at the tip, the base, or anywhere along the length of these protrusions as exemplified in FIG. 16. FIG. 16 demonstrates a representative single protrusion on the skeleton of a representative implantable device showing a magnified illustration of a protrusion (2) and a part of the skeleton (1) of the implantable device, where the protrusion has one or more hooks or other forms of mechanical attachment sites to help attach covering material (3) to it, where the location of the hooks can be at the tip (4) of the protrusion (2) or somewhere along the length (5) of the protrusion (2). Additional adhesive material (6) can be impregnated or added to help better attach the covering material (3).

Another exemplary implantable device of the present invention, as shown in FIG. 17 provides a representative implantable device where the one or more protrusions can be completely, partially, locally or diffusely impregnated or covered with one or more specific materials, wherein optionally, the protrusions have materials with adhesive properties, the said materials with adhesive properties include glue, the said materials with adhesive properties help attach the covering material, the covering material include membrane, mesh, and other material as disclosed herein. Chemicals can also be impregnated in the one or more protrusions and/or the covering material completely, partially, locally or diffusely to prevent clotting, enhance healing of tissue around the device etc. as shown in FIG. 17. FIG. 17 demonstrates a representative single protrusion on the skeleton of a representative implantable device showing a zoomed-up illustration of a protrusion (2) and a part of the skeleton (1) of the implantable device, where the protrusion (2) is covered partially (3) or impregnated or covered diffusely (4) to help attach covering material (5) to it, and the location of said covering can be at the tip (3) of the protrusion (2) or anywhere along the length (4) of the protrusion (2).

In some of the embodiments and examples of the present invention of the implantable device as disclosed herein, wherein the skeleton of the implantable device and its associated one or more protrusions can be made from Nitinol, other memory or non-memory metal, or any other flexible material that can self-expand or self-unfold to any pre-determined shape, when the associated implantable device is released from the delivery catheter.

Another exemplary implantable device of the present invention, as shown in FIG. 18 provides a representative implantable device where the protrusions can be made so that they unfold or expand when an inflatable balloon is used to deploy the implantable device to which they are attached inside the body. The balloon can be positioned inside the body of the implantable device or any other appropriate location within the implantable device itself as shown in FIG. 18. FIG. 18 demonstrates a representative implantable device showing protrusions (3) and the skeleton (2) of the implantable device that is put around an inflatable balloon (1) for delivery where the inflatable balloon (1) is used to expand and/or deploy the implantable device (such as transcatheter aortic valve replacement (TAVR)) comprising its associated protrusions (3) and covering material (4).

In some embodiments and examples of the present invention of the implantable device as disclosed herein, the implantable device and its associated protrusions, are typically inserted into the body by packaging and putting it inside a catheter in a compressed form, then once the catheter is in the appropriate location, the implantable device is released typically under fluoroscopy and/or ultrasound guidance.

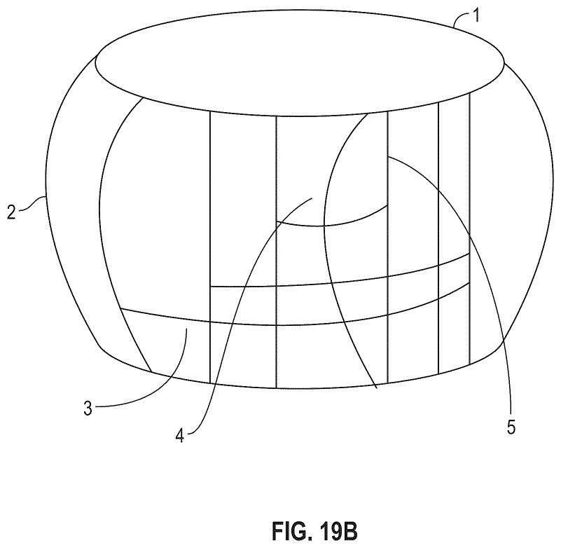

Another exemplary implantable device of the present invention, as shown in FIG. 19 provides a representative implantable device where the skeleton of the implantable device itself may contain at least one independently folding wire or one or more segments which can each flexibly expand, or unfold, into the one or more gaps surrounding the implantable device. In some embodiments and examples of the present invention of the implantable device, the memory metallic segments or components of the skeleton of the implantable expands or unfolds laterally to have an independent motion from the rest of the implantable device to enable better sealing. (Refer FIG. 19A and FIG. 19B). FIG. 19A demonstrates a representative implantable device showing the skeleton (1) of the implantable device, the skeleton (1) comprising components that can have an independent motion (from the rest of the skeleton), where said component can either be an entire part (such as a wire) (2) of the skeleton, or include specific segments (4) connected by connections (3) within the skeleton (1). These wires (2), or segments (4) that are connected by said connections (3) have more independent motion (folding and/or unfolding) unlike other wires, or components of the skeleton (1) linked together with specific connections. FIG. 19B demonstrates a representative implantable device showing the skeleton (1) of the implantable device comprises more flexible wires (2) or segments (4) of more flexible wires, that can independently unfold into gaps around the implantable device, at least partially due to the paucity or lack of connections (3) between such segments of wires, where the connections (3) otherwise limit their independent motion. These flexible segments may sometimes be constrained (5) when there is no gap adjacent to them.

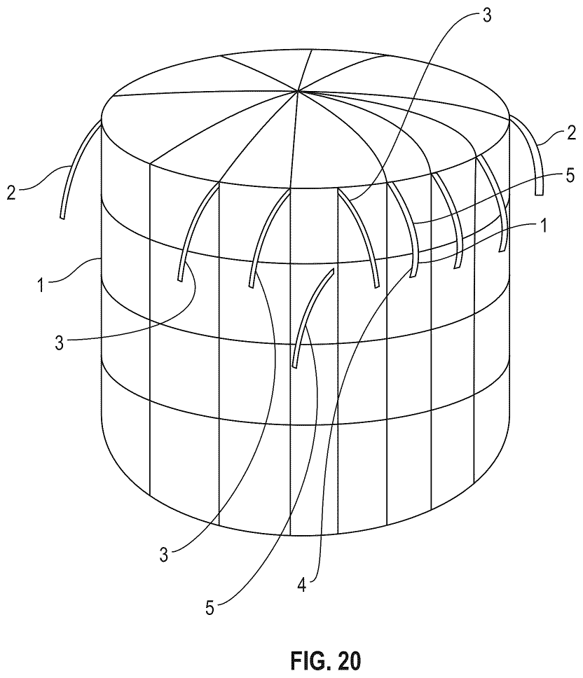

Another exemplary implantable device of the present invention, as shown in FIG. 20 provides a representative implantable device where the protrusions can be individually and/or in any one of the combinations attached to one or multiple wires, parts, and/or other components that constitute the skeleton of the implantable device. In this example and embodiment of the present invention, the protrusions are attached to the skeleton, the main body or the surface of the implantable device, and wherein the protrusions are attached to one or more wires or segments of the implantable device. FIG. 20 demonstrates a representative implantable device showing the skeleton (1) of the implantable device with exemplary protrusions (2), (3), (4), and (5) over the body of the implantable device. Some of the exemplary protrusions (2) are attached to one of the components, parts, or wires of the skeleton (1) of the implantable device. Some of the exemplary protrusions are attached to either two (3) or more (4) wires or components of the skeleton (1) of the implantable device. Some of the protrusions (5) are attached to the body of the implantable device itself.

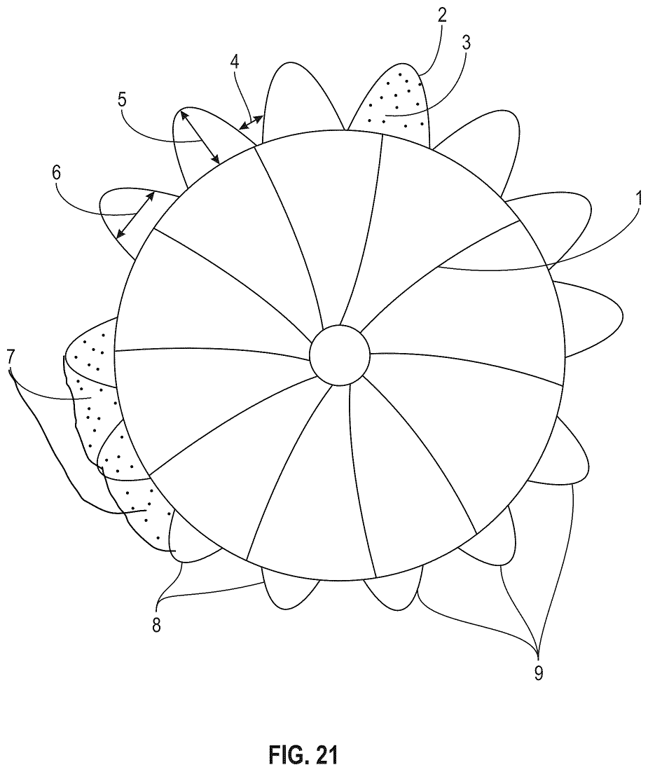

Another exemplary implantable device of the present invention, as shown in FIG. 21 provides a representative implantable device where any two or more of the protrusions can be linked in one of the configurations selected from a group comprising being linked together to other protrusions, linked to the main body of the implantable device, linked to the skeleton of the implantable device, linked to the sites in between protrusions in the attachment sites so that the protrusions can form one or more loops to support the covering material. FIG. 21 demonstrates a representative implantable device showing a top view of an implantable device with its skeleton (1), protrusions (2), and covering material. The protrusions (2) are shaped in a loop-like form with two or more attachments to the body or the skeleton of the implantable device. The covering material can be attached to only one protrusion or protrusion loop at a time (3), or it can extend to cover multiple protrusions (7). The height of each loop (5), the width of each loop (6), and the distance (4) between the protrusions are fixed or variable, and they follow several patterns, for example, long then short (8) pattern, or long, medium and then short loop (9) pattern.

In some of the embodiments and examples of the present invention providing the implantable medical device as disclosed herein, wherein the protrusions can be made from a material selected from a group comprising a memory metal, wherein the memory metal includes Nitinol, other metals or non-metals with similar properties that allow unfolding of such material when released from a compressed form when put inside the delivery catheter for delivery to a particular site in the body for occlusion or prosthetic valve deployment.

In some of the embodiments and examples of the present invention providing the implantable medical device as disclosed herein, wherein the protrusions can assume similar or different shapes, orientations, and/or angles when the implantable device and its associated protrusions are deployed via a delivery catheter.

In some embodiments and examples of the present invention providing the implantable medical device as disclosed herein, wherein the protrusions can have independent motion or can be linked together in groups of 2, 3, 4, 5, or more protrusions, or any combination.

In some embodiments and examples of the present invention providing the implantable medical device as disclosed herein, wherein the tips of the protrusions can be curved to minimize injury to the surrounding tissue when the protrusions are folded or unfolded. Injury to surrounding tissue around implantable devices can also be minimized by making the tips more blunt and not as sharp, such as by making their edges round for instance.

In some embodiments and examples of the present invention providing the implantable medical device as disclosed herein, wherein the length of protrusions can be from 0.001 millimeters up to 1 meter, with a width of 0.001 millimeters up to 1 meter, and a height of 0.001 millimeters up to 1 meter. The thickness of the covering material can be from 0.0001 millimeters up to 1 meter.

In some embodiments and examples of the present invention providing the implantable medical device as disclosed herein, the protrusions can be selected from a configuration selected from a group comprising a protrusion that is narrower, thinner than the skeleton of the implantable device or its components, parts and/or wires.

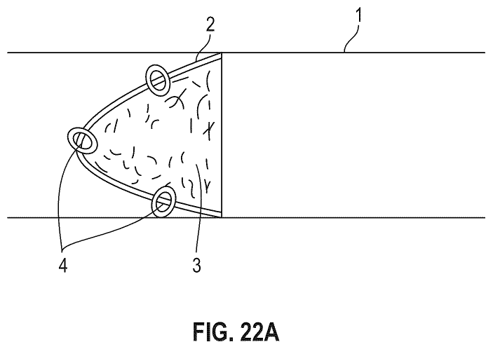

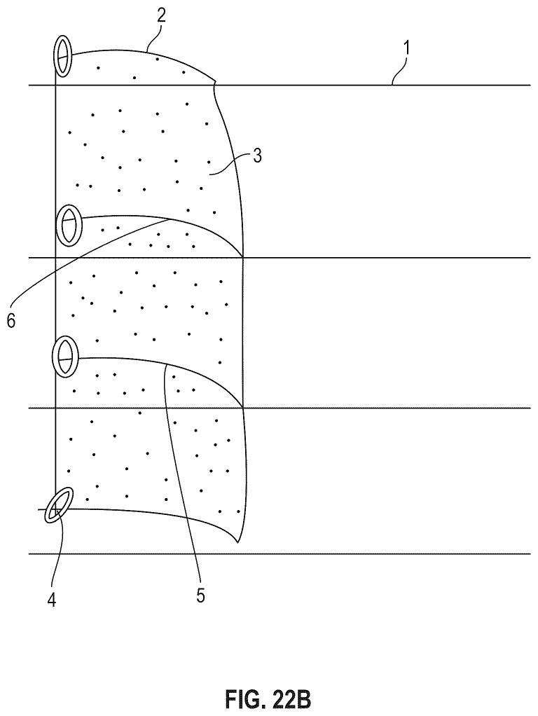

Another exemplary implantable device of the present invention, as shown in FIG. 22 provides a representative implantable device where the two or multiple protrusions can be linked together to form one or more loops or other shapes to better support covering material, membrane, or other flexible covering materials. FIG. 22A demonstrates a representative implantable device showing the skeleton (1) of the implantable device, protrusions (2), and covering material (3). The protrusions (2) form a loop that is attached to two wires or components of the skeleton (1) of the implantable device. The protrusions (2) are attached to the covering material (3) using mechanical attachment sites (4) such as hooks or sutures, or using adhesive material, etc. FIG. 22B demonstrates a representative implantable device showing multiple protrusions (2), (5), and (6) attached to the skeleton (1) of the implantable device, and a covering material (3), where the protrusions (2), (5), and (6) are attached to the covering material (3) using mechanical attachment sites (4) such as hooks or adhesive material as the points of attachment for the covering material.

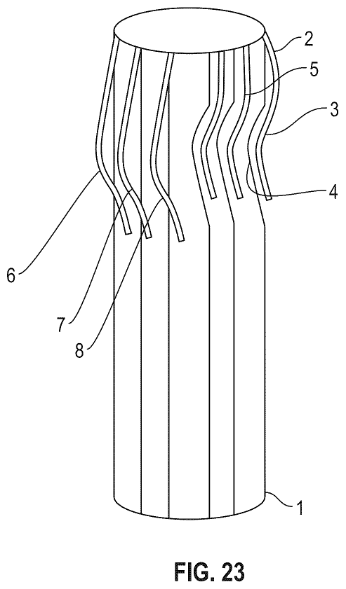

Another exemplary implantable device of the present invention, as shown in FIG. 23 provides a representative implantable device where the protrusions have a complementary shape to each other so that they can stack over each other when the implantable device that the protrusions are attached to is compressed inside a delivery catheter. In some embodiments and examples of the present invention, wherein the protrusions can be curved so that they can stack against a curved segment of the main body or the skeleton of the implantable device or against other protrusions on the implantable device. Protrusions can stack in the same direction, or opposite direction so that the overall size of the implantable device that the protrusions are attached to can be smaller when the device is folded as shown in FIG. 23. FIG. 23 demonstrates a representative implantable device showing the skeleton (1) of the implantable device, and some of the protrusions (2) and (5), where the protrusions (2) and (5) are curved or shaped (3) to stack better (2) and (5) around a specific segment (4) of the implantable device that is curved when the implantable device is compressed inside a delivery catheter. Other protrusions (6), (7), and (8) are curved or shaped so that they can stack better against each other (6), (7), and (8) when the implantable device is compressed inside the delivery catheter.

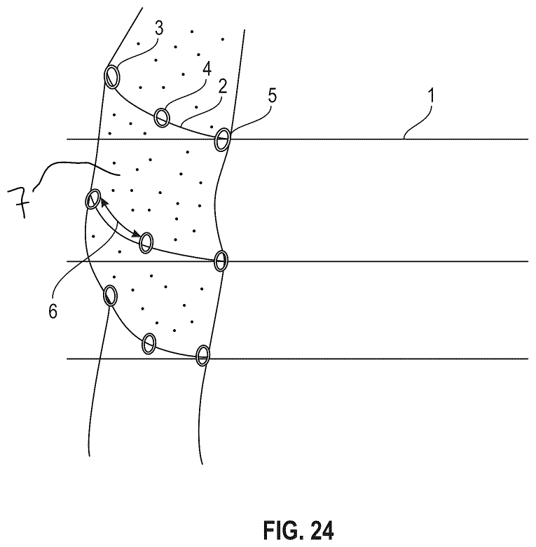

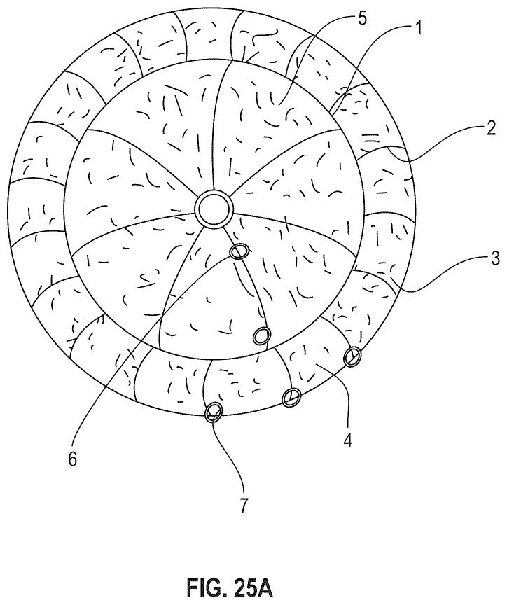

Another exemplary implantable device of the present invention, as shown in FIG. 24 provides a representative implantable device where the covering material as disclosed herein, can be attached and/or sutured to a site on a protrusion, the site selected from a group comprising the center, the tip, at the base, anywhere along the length of each protrusion, or a combination thereof, with one or more points of attachment. FIG. 24 demonstrates a representative implantable device showing the skeleton (1) of the implantable device, and protrusions (2). The protrusions can be attached to the covering material (7) using one or more mechanical attachment sites (3), (4), and (5) such as hooks or adhesive material, etc. These attachment sites can be at the base of the protrusion (5), the tip (3), or anywhere along the protrusion length (4). The distance between these protrusions (6) can be variable or constant.

In another embodiment and example of the present invention providing the implantable medical device as disclosed herein, wherein the covering material can be attached to one or more sites selected from a group comprising the associated implantable device itself, the skeleton, wherein the attachment site can be the periphery, the top or bottom surface or interior aspect of the implantable device as disclosed.

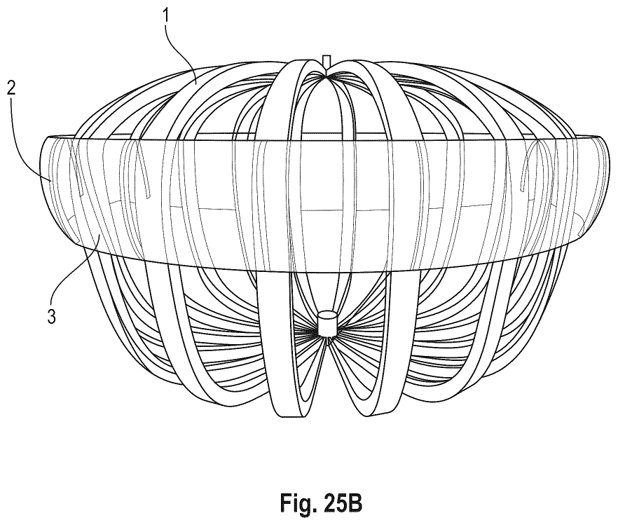

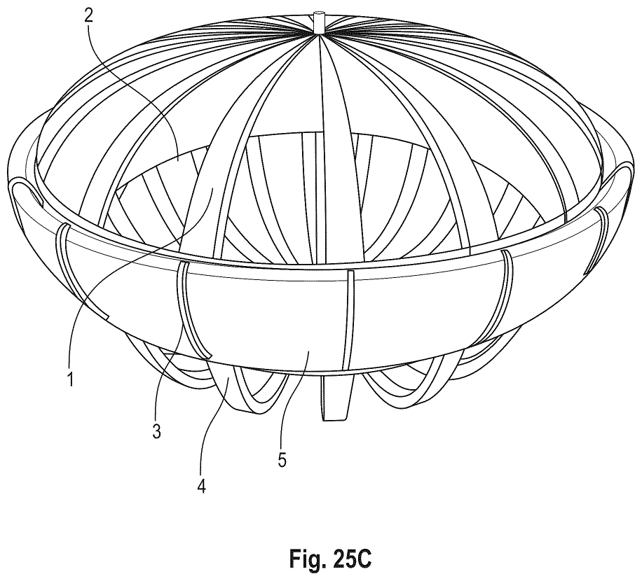

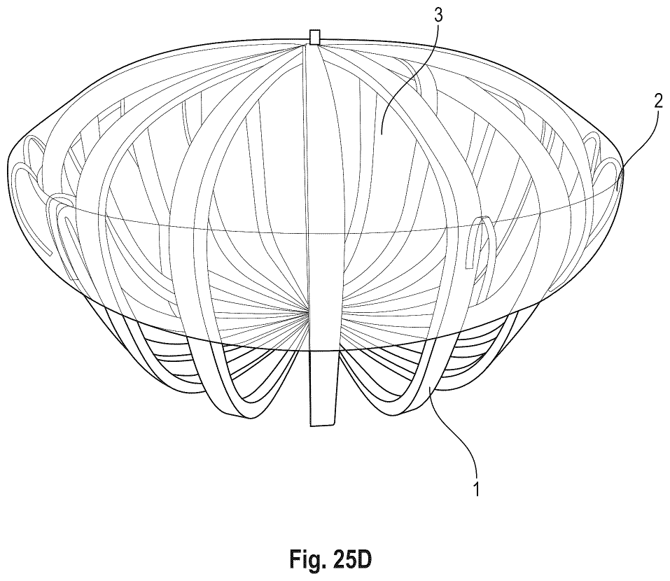

Another exemplary implantable device of the present invention, as shown in FIG. 25 provides a representative implantable device where the covering material can be made using biocompatible or non-biocompatible mesh, porous or nonporous membrane, or other flexible materials. It can be attached to the protrusions, as mentioned earlier, which act like scaffolding to support them. The covering material can also be attached to the skeleton and/or the body of the implantable device itself. The covering material can be attached to both the skeleton and/or the body of the implantable device itself as well as the protrusions. The covering material can help seal the gaps between the implantable device itself and the inner wall of the target aperture and/or organ. It can be made from one piece of material or two or more pieces (or layers). It also can be contiguous with attachments to multiple protrusions and extend out over the skeleton of the implantable device itself (refer FIG. 25A, FIG. 25C, FIG. 25D). The covering material attachment to the protrusions, the skeleton of the implantable device, and/or the main body of the implantable device can be made using sutures, biocompatible glue, mechanical links, etc. In another embodiment and example of the present invention providing the implantable medical device as disclosed herein, wherein the covering material can extend to cover the skeleton and/or the main body of the implantable device in part or total (refer FIG. 25D). In another embodiment and example of the present invention providing the implantable medical device as disclosed herein, wherein the covering material is in a form of a halo-like entity that covers the entire periphery of the implantable device and spans between two or more of the attached protrusions (refer FIG. 25B). FIG. 25A demonstrates a representative implantable device showing the skeleton (1) of the implantable device, protrusions (2), and covering material (3), which is attached to the protrusions. The covering material (3) can be made from one piece that extends over many and/or all the protrusions (4) attached over the skeleton (1) of the implantable device using one or more mechanical attachment sites and/or sutures (7). The covering material (3) can also extend over the body (5) of the implantable device itself and be attached to the skeleton (1) of the implantable device using one or more mechanical attachment sites and/or sutures (6). FIG. 25B demonstrates a representative implantable device showing the skeleton (1) of the implantable device with a covering material (3) attached to multiple and/or all of the protrusions (2). Thus, in this embodiment, the covering material along with the protrusions on the skeleton of the implantable device form a sealing halo around the implantable device. FIG. 25C demonstrates a representative implantable device showing the skeleton of the implantable device, where the skeleton is either covered (1) with a covering material (5) attached to multiple and/or all of the protrusions (3) or the skeleton is uncovered (4). Thus, this embodiment provides a partial covering of the skeleton of the implantable device that forms a distinct form of a sealing halo around a partial skeleton (1) part of the implantable device. Further, the aforesaid sealing halo cover is distinct and separate from another covering material (2) that is used to cover the part of the body of the implantable device itself. FIG. 25D demonstrates a representative implantable device showing the skeleton (1) of the implantable device is partially covered along with part of the body of the implantable device with the same covering material (3) rather than distinct and separate covering materials of the former embodiment, and the same covering material of this embodiment can be attached to parts or whole of both the skeleton and body of the implantable device itself along with the protrusions (2) on them, and thus it can be used to cover the entire device (1).

In some embodiments and examples of the present invention providing the implantable medical device as disclosed herein, the covering material is layered, with stacked layers of similar or various types of materials on top of each other.

In some embodiments and examples of the present invention providing the implantable medical device as disclosed herein, the covering material is used to cover each portion of the implantable device including the skeleton, and periphery of the implantable device.

In another embodiment and example of the present invention providing the implantable medical device as disclosed herein, the covering material is attached to the implantable device at a site selected from a group comprising of one or more of the protrusions, the main body, the skeleton, or any combination thereof.

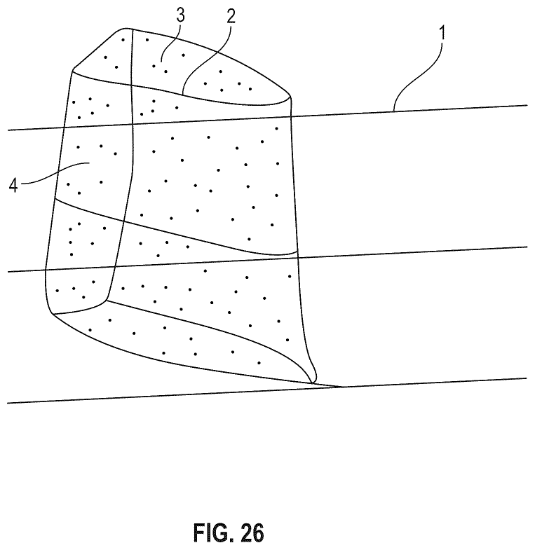

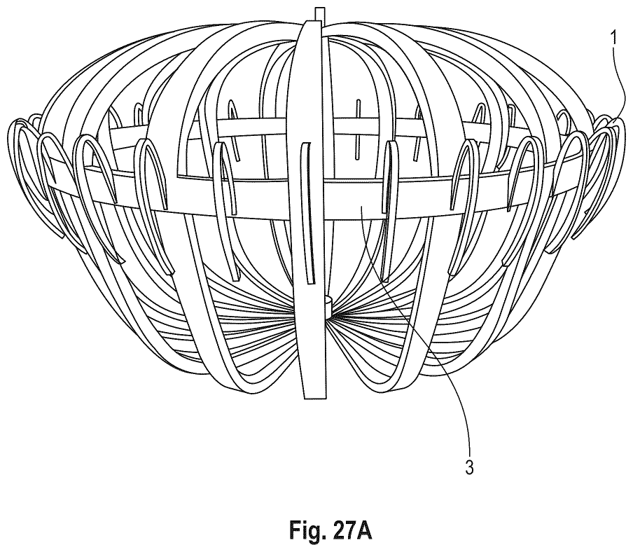

Another exemplary implantable device of the present invention, as shown in FIG. 26 provides a representative implantable device where the covering material comprises an expandible material, wherein the expandible material comprises a foam or other filling material to further fill any gap in the space surrounding the implantable device. FIG. 26 demonstrates a representative implantable device showing the skeleton (1) of the implantable device, and protrusions (2), where the protrusions are attached to a covering material (3). The covering material (3) contains a foam-like material (4) that can help further cover the gaps surrounding the implantable device.

In the present invention as disclosed herein, the tethering of the covering material to the protrusions can be redundant to allow further flexibility when the protrusions move given their independent motion.

In the present invention as disclosed herein, the covering material can be redundant to allow more freedom of movement between the associated, i.e., attached or linked protrusions.

In the present invention as disclosed herein, multiple independent or semi-independent protrusions with same or variable lengths, i.e., random variation in lengths of protrusions, or patterned variation in lengths, where the lengths of adjacent follow a certain pattern, with an attached covering material. The covering material can also be attached to the implantable device. The protrusions and the covering material can form a halo around the implantable device. The halo attached to the implantable device can be compressed alongside the implantable device inside a delivery catheter, then it can unfold to cover any gap around the implantable device once inside the body at the specific site.

In the present invention as disclosed herein, the covering material can be attached to any part of the impalpable device or its entire surface including bottom, top or interior of the surface.

In the present invention as disclosed herein, the covering material can be attached to any one or combination of side(s) of the protrusions (side, top, or bottom etc.).

In the present invention as disclosed herein, the implantable device may be deployed using an internal balloon catheter, while the protrusions and the attached covering material to the protrusions will unfold on their own based on the memory properties of the protrusions and the presence or absence of a gap between the deployed implantable device and the organ walls surrounding it. For example, a TAVR is deployed using a balloon expansion, then the attached protrusions and attached covering material and/or sealing halo will unfold spontaneously based on the memory properties of the protrusions.

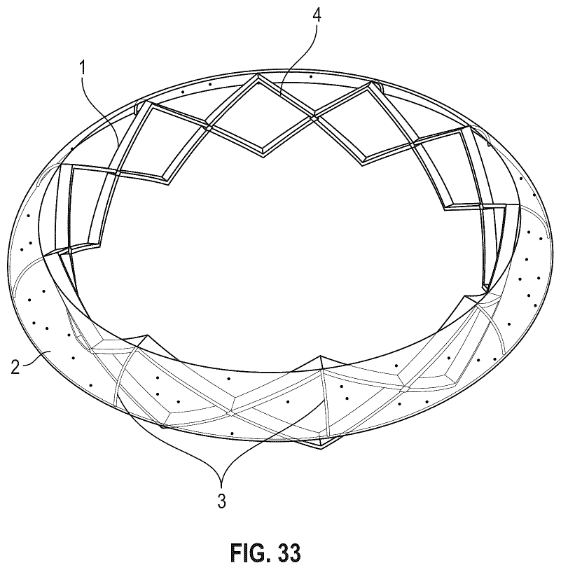

Another embodiment and example of the present invention providing the implantable medical device as disclosed herein, wherein the protrusions can be attached to a flexible ring made from a memory metal such as Nitinol or other flexible material. The flexible sealing ring can be stent-like (refer to FIG. 33). FIG. 33 demonstrates a representative exemplary sealing halo unit in relation to an exemplary implantable device showing a sealing halo (1), where the protrusions (3) can be attached to a flexible stent-like ring (4), which can be attached to an implantable device, and a covering material (2) can also be attached to these protrusions (3) and/or the stent-like ring (4). A combination of all of these (1), (2), (3), (4) is another embodiment of a sealing halo unit of the present invention that helps seal gaps around the implantable device, such as a left atrial appendage (LAA) occluder, transcatheter aortic valve replacement (TAVR), etc. The sealing halo unit can be attached to or built into the implantable device, such as a TAVR, LAA occluder, or any other implantable medical device. The sealing halo unit can also be fused to the skeleton of the implantable device, such as an LAA occluder, TAVR, or any other implantable medical device. The covering material can also be attached to these protrusions and/or the ring. A combination of all of these can form a unit called a sealing halo unit that helps seal gaps around the implantable device.

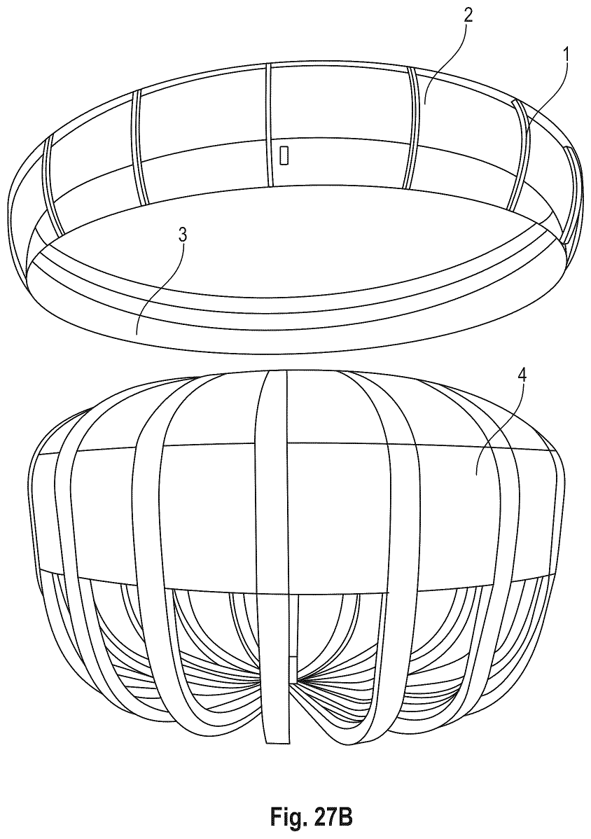

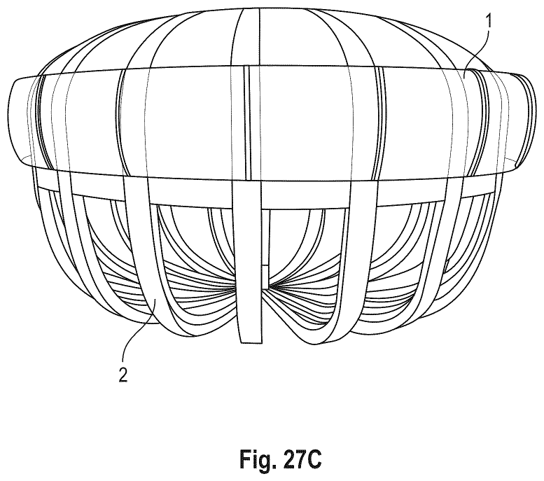

An exemplary sealing halo unit in relation to an exemplary implantable device of the present invention, as shown in FIG. 27 provides a representative implantable device where this unit can be attached or built-in as a part of any implantable device such as LAA occluder, TAVR, etc. (refer to FIG. 27A and FIG. 27C). Typically, the sealing halo unit is attached or fused or built to be attached to the skeleton of the implantable device (refer to FIG. 27B). FIG. 27A demonstrates a representative exemplary sealing halo unit in relation to an exemplary implantable device showing an implantable device embodiment, where the protrusions (1) are attached to a flexible ring (3) which is built into the implantable device itself. FIG. 27B demonstrates a representative exemplary sealing halo unit in relation to an exemplary implantable device showing an embodiment of the present invention exemplified a separate sealing halo unit comprising the protrusions (1) and the flexible ring (3) both covered by a covering material (2) (top ring-like structure), where a combination of all of these (1), (2), and (3) is referred to as the sealing halo unit that helps seal gaps around the implantable device, and it is used to be fused to the skeleton (4) of the implantable device (bottom ring-like structure). Here, the sealing halo unit can be either attached to or built into the implantable device such as a transcatheter aortic valve replacement (TAVR) or left atrial appendage (LAA) occluder (4). FIG. 27C demonstrates a representative exemplary sealing halo unit in relation to an exemplary implantable device showing another embodiment of the implantable device of the present invention, where the protrusions (1) are attached to a flexible ring (3) which is attached to the implantable device, and one or more covering material (2) is attached to these protrusions (1) and/or the ring (3).

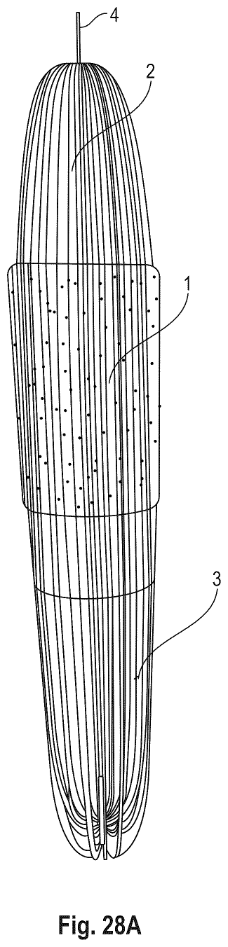

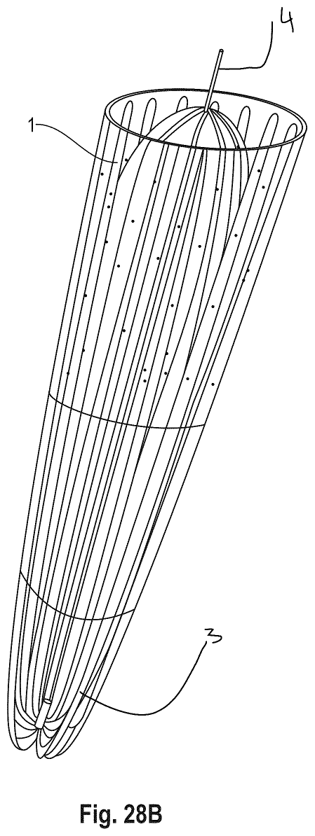

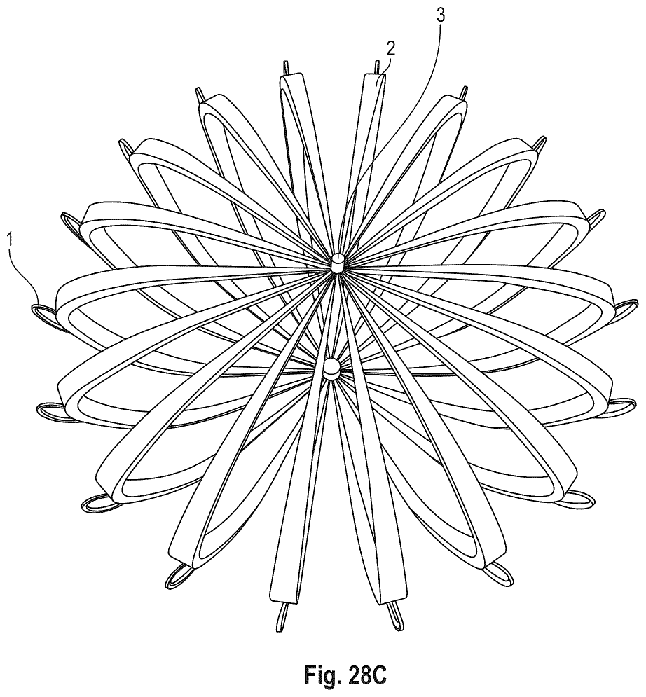

Another exemplary implantable device of the present invention, as shown in FIG. 28 provides a representative implantable device where the protrusions, the covering material, and/or the sealing halo unit can be refolded back if the location of the associated implantable device is not optimal during delivery. In some embodiments of the present invention, the protrusions, the covering material and/or the sealing halo unit can be refolded by pulling the associated implantable device which results in pulling of the associated protrusions, the covering material and/or the sealing halo back into the delivery catheter. Then the location of the catheter and/or orientation can be optimized before another attempt to deploy them is done. The protrusions, covering material and/or sealing halo unit can be refolded in the original orientation or in some other embodiments in the opposite orientation to enable pulling back into the catheter. The pulling mechanism helps pull the main implantable device as well as associated protrusions, covering material, and/or sealing halo (refer to FIG. 28A, FIG. 28B, FIG. 28C). FIG. 28A demonstrates a representative implantable device showing an implantable medical device, where the protrusions, covering material and/or sealing halo unit (1) can be refolded back in the same direction as the original compressed state before deployment. In the implantable device, the skeleton (3) of the implantable device and covering (2) of the implantable device are also displayed along with a pulling rod mechanism (4) which is used to pull all these components back into the delivery catheter. FIG. 28B demonstrates a representative implantable device showing another embodiment of the implantable medical device of the present invention shown with protrusions, covering material and/or sealing halo unit (1) that can be refolded back in the opposite direction as the original compressed state shown in panel (A). In another embodiment and example of the present invention providing the implantable medical device as disclosed herein, wherein a pulling mechanism and/or a rod can be attached to the skeleton of the implantable device. It can help in pulling the entire implantable device and its associated sealing halo and/or protrusions and attached covering material back into the delivery catheter as well as help in advancing them into position during delivery (refer to FIG. 28C). FIG. 28C demonstrates a representative implantable device showing another embodiment of the implantable medical device of the present invention is shown with protrusions (1) attached to the skeleton (2) of the implantable device. The pulling rod mechanism (3) is also attached to the skeleton (2) of the implantable device. It can help in pulling the entire implantable device and its associated sealing halo unit (and/or protrusions and attached covering material) back into the delivery catheter as well as it helps in advancing them into position during delivery.

In some embodiments and examples of the present invention as disclosed herein, the protrusions can expand at a slower pace and/or at a lower force than the implantable device that they are attached to.

Unfolding of the protrusions and the associated covering material and/or sealing halo can occur sideways, diagonally, or vertically when compared with the orientation of the implantable device.

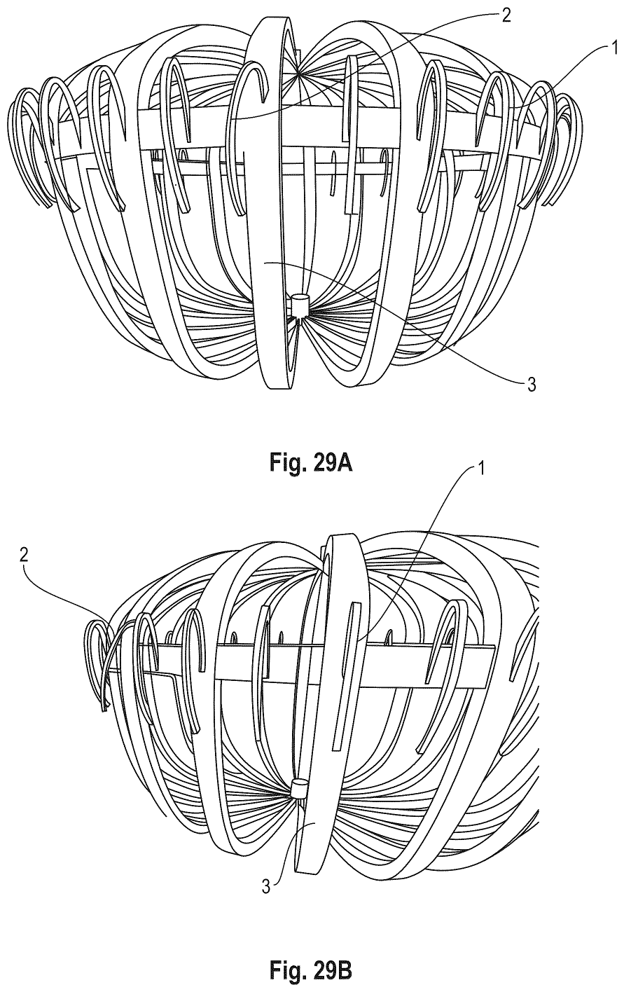

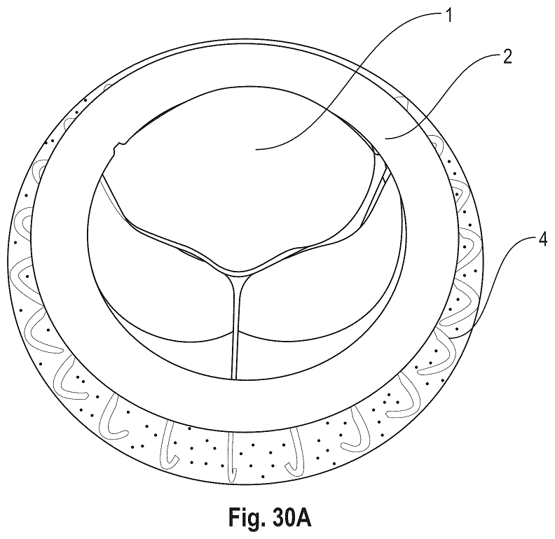

Another exemplary implantable device of the present invention, as shown in FIG. 29 provides a representative implantable device where the protrusions can swivel sideways when they unfold. In another embodiment of the present invention providing the implantable medical device as disclosed herein, wherein the protrusions unfold parallel to the implantable device, or in any other orientation when compared to the implantable device (refer to FIG. 29A and FIG. 29B). FIG. 29A demonstrates a representative implantable device showing an implantable medical device, where the protrusions (1) can swivel sideways (2) when they unfold when compared to the implantable device and/or its skeleton (3). FIG. 29B demonstrates a representative implantable device showing another embodiment of the implantable medical device of the present invention with protrusions (1) that can swivel parallel (2) when they unfold when compared to the implantable device and/or its skeleton (3).

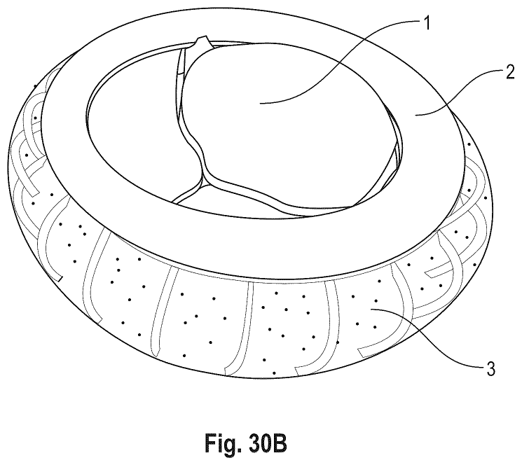

Another exemplary implantable device of the present invention, as shown in FIG. 30 provides a representative implantable device where the implantable device is TAVR, the length of the halo comprising the protrusions and the covering material associated with the protrusions does not interfere with the coronary arteries ostia. The protrusions and/or the halo seal unit can be attached to the outer aspect of the annulus of the TAVR or other types of transcatheter prosthetic valves (refer to FIG. 30A and FIG. 30B). FIG. 30A demonstrates a representative exemplary sealing halo unit in relation to an exemplary implantable device showing the top view of an implantable device where protrusions (4) and/or sealing halo unit (3) are attached to the outer aspect of the annulus (2) of the implantable device which here is a transcatheter aortic valve replacement (TAVR) (1). FIG. 30B demonstrates a representative exemplary sealing halo unit in relation to an exemplary implantable device showing the side view of an implantable device where protrusions (4) and/or sealing halo unit (3) are attached to the outer aspect of the annulus (2) of the implantable device which here is a transcatheter aortic valve replacement (TAVR) (1).

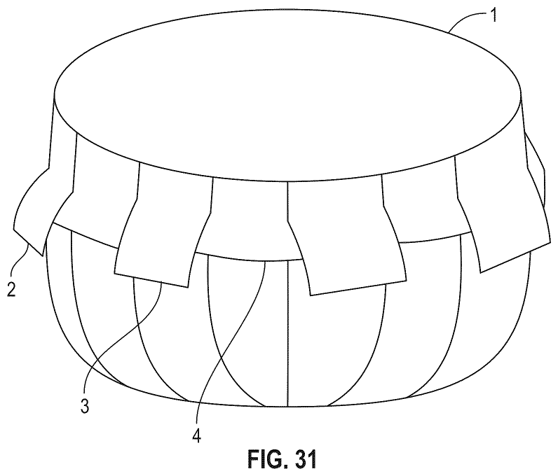

Another exemplary implantable device of the present invention, as shown in FIG. 31 provides a representative implantable device where implantable device is TAVR, wherein the protrusions may be linked to each other through interconnection to achieve a synchronous movement when they fold or unfold. The links can be made from the same material as the protrusions such as memory metal or other flexible material. The links can be attached to the tips of the protrusions or anywhere along their lengths, all the links can form an outside ring that is attached to some or all of the protrusions (refer to FIG. 31). FIG. 31 demonstrates a representative exemplary implantable device showing the skeleton (1) of the implantable device, along with protrusions (2) that have independent motion from the skeleton (1) of the implantable device. Interconnections or links can be made or built between the protrusions (2) and can be attached anywhere along the length of the protrusions whether the tips (3) of the protrusions, or the body (4) of the protrusions.

In the present disclosure, the protrusions may have independent and/or semi-independent and/or fixed movement from each other, and also from the implantable device itself.

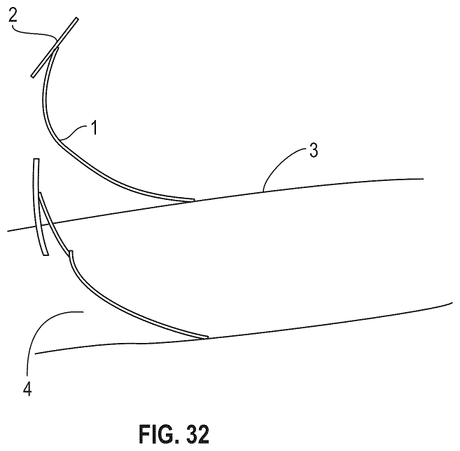

Another exemplary implantable device of the present invention, as shown in FIG. 32 provides a representative implantable device where the implantable device is TAVR, or other transcatheter prosthetic valves, wherein the protrusions have additional extension, said extension comprising an arch extension to enable better and smoother expansion. These extensions can be made from memory metal or other flexible materials, they also can be attached to a covering material (refer to FIG. 32). FIG. 32 demonstrates a representative exemplary implantable device showing a magnified illustration of an implantable device with protrusions (1) that may have an additional extension (2) such as an arch extension to enable better and smoother expansion. These extensions (2) can be made from memory metal or other flexible materials, these extensions (2) can also be attached to a covering material (4) along the skeleton (3) of the implantable device.

In another embodiment and example of the present invention providing the implantable medical device as disclosed herein, wherein the implantable device is TAVR, or other transcatheter prosthetic valves, wherein the covering material is made of a material selected from a group comprising of polyester fibric, polyethylene, nylon, a metal alloy mesh, pTFE polyethylene terephthalate fabric, or other porous or semi-porous materials.

In another embodiment and example of the present invention providing the implantable medical device as disclosed herein, wherein the implantable device is selected from a group comprising of an existing LAA occluder, a future LAA occluder, a single LAA occluder device unit including a Watchman, or Conformal device, articulating LAA occluder, LAA occluder that consists of multiple units which is similar to an Amulet device which can have added protrusions and covering materials, or a sealing halo to enable better sealing.

In another embodiment and example of the present invention providing the implantable medical device as disclosed herein, wherein the implantable device is a self-expanding TAVR, or other transcatheter prosthetic valves, having a sealing halo attached or built into the annulus of the TAVR, or other transcatheter prosthetic valves, to enable better sealing. The sealing halo has an independent motion from the TAVR, or other transcatheter prosthetic valves, themselves.

The above-mentioned embodiments can be used, individually or in any combination(s), as a part of any implantable device or used as a modification to the device. These devices can be any implantable medical device or other occluder or functional device used in other industry such as oil industry.

It will be apparent to those skilled in the art that various modifications and variations can be made in the practice of the present invention without departing from the scope or spirit of the invention. Other embodiments of the invention will be apparent to those skilled in the art from considering of the specification and practice of the invention. It is intended that the specification and examples be considered as exemplary only, with a true scope and spirit of the invention being indicated by the following claims.

What is claimed is:

1. An implantable device, the implantable device comprises:
a main body;
a skeleton;
one or more protrusions; and
a covering material,
wherein the main body is selected from a group comprising an occluder and a prosthetic valve,
wherein the occluder is selected from a group comprising occluders to occlude a cavity, aperture, vessel, tube, channel, conduit, and duct;
wherein the prosthetic valve is a valve that directs and regulates liquid flow in a certain direction,
wherein the skeleton and the covering material support the main body of the implantable device and create and maintain its shape,
wherein the covering material is attached to the implantable device,
wherein the covering material is a biocompatible covering material,
wherein the one or more protrusions are attached or linked to the covering material, and
wherein the one or more protrusions, and the covering material, expand into gaps around the implantable device to fill in the gaps not filled by the main body and skeleton of the implantable device.

2. The implantable device of claim 1, wherein the implantable device is a medical device, wherein the medical device is an occluder for occluding a cavity, aperture, or vessel in the body of an animal, wherein the animal is a mammal.

3. The implantable device of claim 1, wherein the implantable device is a medical device, wherein the medical device is a prosthetic valve, wherein the prosthetic valve is a replacement for a native valve in the body of an animal as an artificial valve, wherein the animal is a mammal.

4. The implantable device of claim 1, wherein the implantable device is a non-medical device, wherein the non-medical device is an occluder for occluding a tube, channel, conduit, or duct.

5. The implantable device of claim 1, wherein the implantable device is a non-medical device, wherein the medical device is a prosthetic valve, wherein the prosthetic valve is a replacement for a faulty valve in a tube, channel, conduit, or duct that regulates the flow of a liquid through said tube, channel, conduit, or duct.

6. The implantable device of claim 1, wherein the implantable device is a medical device, wherein the medical device enables better sealing and anchoring of implantable medical devices selected from a group of devices comprising left atrial appendage (LAA) occluders, Transcatheter Aortic Valve Replacement (TAVR), Transcutaneous Mitral Valve Replacement (TMVR), Transcatheter Tricuspid Valve Replacement (TTVR), Transcatheter Pulmonic Valve Replacement (TPVR), Atrial Septal Occluders, Patent Foramen Ovale Occluders, and vascular plugs.

7. The implantable device of claim 1, wherein the one or more protrusions are selected from a group comprising protrusions that are flexible and independent, semi-flexible and independent, semi-independent, and linked and built-in as part of the main body and/or skeleton of the implantable device.

8. The implantable device of claim 1, wherein the covering material is selected from a group of materials comprising porous, semi-porous, or non-porous membranes, a mesh made of porous, semi-porous, or non-porous materials, and other flexible or nonflexible materials that can provide additional sealing to the implantable device.

9. An implantable device for the treatment of atrial fibrillation, the implantable device comprises:
a main body;
a skeleton;
one or more protrusions; and
a covering material,
wherein the implantable device for the treatment of atrial fibrillation is a left atrial appendage (LAA) occluder,
wherein the skeleton and the covering material support the main body of the implantable device and create and maintain its shape,
wherein the covering material is attached to the implantable device,
wherein the covering material is a biocompatible covering material,
wherein the one or more protrusions are attached or linked to the covering material, and
wherein the one or more protrusions, and the covering material, expand into gaps around the implantable device to fill in the gaps not filled by the main body and skeleton of the implantable device.

10. The implantable device for the treatment of atrial fibrillation of claim 9, wherein the skeleton comprises a memory metal, wherein the memory metal is selected from a group comprising Nitinol, shape-memory alloys, and other flexible materials, and wherein the shape-memory alloys include copper-aluminum-nickel and nickel-titanium (NiTi), and other alloys containing a metal selected from a group comprising zinc, copper, gold, and iron.

11. The implantable device for the treatment of atrial fibrillation of claim 9, wherein the skeleton and the biocompatible covering material are linked together to allow the main body and the biocompatible covering material to expand together as a unit with limited independent movement of the LAA occluder.

12. The implantable device for the treatment of atrial fibrillation of claim 9, wherein the one or more protrusions are selected from a group comprising protrusions that are flexible and independent, semi-flexible and independent, semi-independent, and linked and built-in as part of the main body and/or skeleton of the implantable device.

13. The implantable device for the treatment of atrial fibrillation of claim 9, wherein the covering material is selected from a group of materials comprising porous, semi-porous, or non-porous membranes, a mesh made of porous, semi-porous, or non-porous materials, and other flexible or nonflexible materials that can provide additional sealing to the implantable device.

14. An implantable device for the treatment of an abnormal valve in the body of an animal, the implantable device comprises:
a main body;
a skeleton;
one or more protrusions;
a covering material;
an annulus; and
valve leaflets,
wherein the implantable device is a prosthetic valve that directs and regulates liquid flow in a certain direction and it is a replacement for a native valve in the body of an animal as an artificial valve,
wherein the implantable device is a Transcatheter Aortic Valve Replacement (TAVR), a Transcatheter Mitral Valve Replacement (TMVR), Transcatheter Tricuspid Valve Replacement (TTVR), Transcatheter Pulmonic Valve Replacement (TPVR),
wherein the animal is a mammal,
wherein the skeleton and the covering material support the main body of the implantable device and create and maintain its shape,
wherein the covering material is attached to the implantable device,
wherein the covering material is a biocompatible covering material,
wherein the one or more protrusions are attached or linked to the covering material, and
wherein the one or more protrusions, and the covering material, expand into gaps around the implantable device to fill in the gaps not filled by the main body and skeleton of the implantable device.

15. The implantable device for the treatment of an abnormal valve in the body of an animal of claim 14, wherein the skeleton comprises a memory metal, wherein the memory metal is selected from a group comprising Nitinol, shape-memory alloys, and other flexible materials, and wherein the shape-memory alloys include copper-aluminum-nickel and nickel-titanium (NiTi), and other alloys containing a metal selected from a group comprising zinc, copper, gold, and iron.

16. The implantable device for the treatment of an abnormal valve in the body of an animal of claim 14, wherein the skeleton and the biocompatible covering material are linked together to allow the main body and the biocompatible covering material to expand together as a unit with limited independent movement of the TAVR, TMVR, TTVR, or TPVR.

17. The implantable device for the treatment of an abnormal valve in the body of an animal of claim 14, wherein the one or more protrusions are selected from a group comprising protrusions that are flexible and independent, semi-flexible and independent, semi-independent, and linked and built-in as part of the main body and/or skeleton of the implantable device.

18. The implantable device for the treatment of an abnormal valve in the body of an animal of claim 14, wherein the covering material is selected from a group of materials comprising porous, semi-porous, or non-porous membranes, a mesh made of porous, semi-porous, or non-porous materials, and other flexible or nonflexible materials that can provide additional sealing to the implantable device.

19. A sealing halo unit, comprising:
an attachment ring;
one or more protrusions; and
a covering material,
wherein the one or more protrusions and the covering material are attached or built onto the attachment ring to provide the sealing halo unit,
wherein the sealing halo unit provides better sealing and/or better anchoring of the implantable device to a specific surface,
wherein the sealing halo unit is a flexible halo that is further fused to or attached to or built as part of an implantable device around the outside of the implantable device, and
wherein the one or more protrusions, the covering material, and the attachment ring independently expand into gaps around the implantable device to fill in the gaps not filled by the main body and skeleton of the implantable device to provide better sealing and/or better anchoring.

20. The sealing halo unit of claim 19, wherein the one or more protrusions support the attached covering material to maintain its structure and are selected from a group comprising protrusions that are flexible and independent, semi-flexible and independent, semi-independent, and linked and built-in as part of attachment ring, and wherein the covering material is selected from a group of materials comprising porous, semi-porous, or non-porous membranes, a mesh made of porous, semi-porous, or non-porous materials, and other flexible or nonflexible materials that can provide additional sealing to the implantable device.
